SEQUENCE LISTING (46 sequences)

SEQ ID NO: 1 - DNA, 1269 bp, Bacillus subtilis
atgttcgtgaatacagttatagcgtttatcattattttcggaacgctcgttttcttccatgaactgggccatttattgctagcccaaagagcgggaattctctgccgtgaatttgcgatcggcttcggtccaaagattttttctttcaaaaaaaatgaaacagtttatacgatcaggctgcttccggtcggcggatttgttcgtatggccggcgaagatccggaaatgattgaggtgaaacccggatacacggtcgggcttctgtttaataaggaagatcaagttgagaaagtcatcatcaatcaaaaggaaaaatatccggatgctttagtcattgaagtggaaacagcggatctagagcatgacatgaagatcaccggttatgaacaggggaaagaggacgaactttccagctttactgtcagcgaaacatccttttttattgtagacggagaagaagtgcagattgcgccgtataatcgccaatttggttccaaacctgtatggcagcggattaaagcaattgctgcagggccgattatgaactttattttagcttacgtcattttagtgatgcttgggctgattcaaggcgtaccgtcaaatgaacctatgctcgggcagctgacagacaatggacgggcggctgaagcagggctaaaagaaggggattatatccaaagcattaacggagagaaaatgaggtcttggactgacattgtctccgctgtaaaagaaaacccggagaaagaaatggacgttgcagtaaaaagagataacaaaacgcttcatatttcggtgactccggaagctgttaaagatgagaacaaaaaaacaatcggacgtttcggttcctatgcgccgactgaaaaaggcgtactctcagcggttgcttacggcgcgacatcaacagttgatgtcaccaaagccattttaaccaatctgagcaaattagtaacaggccaatttaaactcgatatgctgtcaggtcctgtcggcatatatgacatgacagaccaagtggcgaaaacagggatcgtgaacttatttcagtttgcggcgtttttaagcattaaccttgggattgtcaacctgcttccgattccggcacttgacggaggaagactgttgtttctatttattgaagcgattcggggcaaaccgattaacagggaaaaagaagcatttgttgtgtttatcggagtagctttcttaatgcttcttatgctggttgtcacatggaacgatatccagcggctgtttttgtaa

SEQ ID NO: 2 - protein, 422 aa, Bacillus subtilis
MFVNTVIAFIIIFGTLVFFHELGHLLLAQRAGILCREFAIGFGPKIFSFKKNETVYTIRLLPVGGFVRMAGEDPEMIEVKPGYTVGLLFNKEDQVEKVIINQKEKYPDALVIEVETADLEHDMKITGYEQGKEDELSSFTVSETSFFIVDGEEVQIAPYNRQFGSKPVWQRIKAIAAGPIMNFILAYVILVMLGLIQGVPSNEPMLGQLTDNGRAAEAGLKEGDYIQSINGEKMRSWTDIVSAVKENPEKEMDVAVKRDNKTLHISVTPEAVKDENKKTIGRFGSYAPTEKGVLSAVAYGATSTVDVTKAILTNLSKLVTGQFKLDMLSGPVGIYDMTDQVAKTGIVNLFQFAAFLSINLGIVNLLPIPALDGGRLLFLFIEAIRGKPINREKEAFVVFIGVAFLMLLMLVVTWNDIQRLFL

SEQ ID NO: 3 - DNA, 159 bp, Artificial Sequence
  OLIGO
taagaaaagtgattctgggagagccgggatcacttttttatttaccttatgcccgaaatgaaagctttatgacctaattgtgtaactatatcctattttttcaaaaaatattttaaaaacgagcaggatttcagaaaaaatcgtggaattgatacacta

SEQ ID NO: 4 - DNA, 568 bp, Bacillus subtilis
aattcctccattttcttctgctatcaaaataacagactcgtgattttccaaacgagctttcaaaaaagcctctgccccttgcaaatcggatgcctgtctataaaattcccgatattggcttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaataatactcacataaggtggt

SEQ ID NO: 5 - DNA, 58 bp, Bacillus subtilis
acagaatagtcttttaagtaagtctactctgaatttttttaaaaggagagggtaaaga

SEQ ID NO: 6 - protein, 11 aa, Artificial Sequence
  12-amino acid residue active site consensus sequence, which aligns with amino acid residues 16-26 of SEQ ID NO 2.
  ACT_SITE (1)..(11): 11-amin acod residue consensus sequence qhich aligns to amino acid residues 16-26 of SEQ ID NO 2.
LVFFHELGHLL

SEQ ID NO: 7 - protein, 5 aa, Artificial Sequence
  Active site consnesus sequence, aligns to amino acid residues 20-24 of SEQ ID NO2
  ACT_SITE (1)..(5): 5-amino acid consensus sequence which aligns with amino acid residues 20-24 of SEQ ID NO 2.
  MISC_FEATURE (3)..(3): Xaa is any amino acid
  MISC_FEATURE (4)..(4): Xaa is any amino acid
HEXXH

SEQ ID NO: 8 - DNA, 83 bp, Artificial Sequence
  oligo
cgagctcgaattcactggccgtcgggatacgtcaattcaatactcacataaggtggtacgaaaagtaaatcaatcagaggtgc

SEQ ID NO: 9 - DNA, 17 bp, Artificial Sequence
  oligo
gatcgtacggcgcaacg

SEQ ID NO: 10 - DNA, 19 bp, Artificial Sequence
  oligo
gctcttcaaggcgaacagg

SEQ ID NO: 11 - DNA, 19 bp, Artificial Sequence
  oligo
cttcaacgctaactttgag

SEQ ID NO: 12 - DNA, 20 bp, Artificial Sequence
  oligo
ctgtccgttccagtgtacgg

SEQ ID NO: 13 - DNA, 40 bp, Artificial Sequence
  oligo
cgacctgcaggcatgcaagctctcgctttcatcctttccg

SEQ ID NO: 14 - DNA, 78 bp, Artificial Sequence
  oligo
cgagctcgaattcactggccgtcgcaaagagaaactcggaaaggatgaaagcgagttctttataccgtgatgcctcag

SEQ ID NO: 15 - DNA, 26 bp, Artificial Sequence
  oligo
ggtctgtcattcaatttagactccag

SEQ ID NO: 16 - DNA, 15 bp, Artificial Sequence
  oligo
cgcacgggcacgatg

SEQ ID NO: 17 - DNA, 1040 bp, Bacillus subtilis
tcgctgaggttctccctgaatccatgaaaaacgccctttctgaaataccggaacaacaatggctggaaatcgaagaagtcagaattcggatcaaccgccctgttgaattgattcgaagaggacaacctgtttatttgtcctatgcgggcacggccgaggatgcccatctgatcctgagccggctcagcaactatagtatgtatacacttgaagaagagctgaagaaagggtatgtcacgatcagaggcggccacagagtcggactggcgggcagagtcattacggaaaacggaggtgtgaaaggcctccgtgacatcgcatcttttaatatacgaattgcccggcaaaagctggggattgcggagccgctgcttccatacttatatcaaaacagctggctgaatacgctgattatcggaccgccccaaaccggcaaaacaacacttctccgggaccttgcgaggctctcaagcaccggcaaaaaaaacatgctgcccgtaaagacagggatcgtcgatgaacggtcagagattgccggatgtcttcgcggtattccgcagcatcaatttggtcagagaattgacgtgttagacgcctgtccaaaagctgaagggctgatgatgatgattcgatccatgagccctgaggtgatgattgtcgatgaaatcgggcgtatggaagatacagatgcacttctggaggcgctgcatgccggtgtttcagtaatcgtatcggcgcatggctggagtatatccgacctgatgaagcggccttcattgaagcgtttgtgggaagaaagagcctttgaccgctatttggaattatcaagagcaaaagggccgggcacagtgagccaaatttatgacaaagacggaaatgtgctgtctaggacgacgggcgtgaaaacatgctgaagctgctgggcgctgtattcattgtggtagcgacaacatggacaggttttgagatggcgaagatttatacagaacggccgcggcaaatccgccagctgcgtgcggcgcttcaatccctcgaggctg

SEQ ID NO: 18 - DNA, 52 bp, Artificial Sequence
  oligo
cgagaaaggaagggaagaaagcgaaatcgctgaggttctccctgaatccatg

SEQ ID NO: 19 - DNA, 22 bp, Artificial Sequence
  oligo
cagcctcgagggattgaagcgc

SEQ ID NO: 20 - DNA, 1131 bp, Artificial Sequence
  SpcR
gcgcttcaatccctcgaggctgtcgacggtatcttattgcggatccataacttcgtataatgtatgctatacgaagttatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgtctagataacttcgtataatgtatgctatacgaagttatgcggccgccatatgcatcctaggcc

SEQ ID NO: 21 - DNA, 981 bp, Bacillus subtilis
aagcttcccagcgcgcatatattctagaagaaatggctgtccaactaaaaaagaaagcggaggagagattcagtcatgatgaatataaagtaggccgcatcaaactcacggcaggagaaaaggtagattcagaagaggatattaaaacaatcagcgtgtatatggccccgtcttctgaaaaaacggtccaaacggtcgcgccggtccacattgatacagatcatgcatacgtaacaaaagaagcagccgaacaaaaagaagccaaacagatacaaacgcagctcgcggacatttgggaaatcggaagtgagaaaattacggttcatatggaaggcggggagagtgtcggcaatgaataaaaacggattatggaatgtattgaaaaagcagtttcttccgggacaaacgaaggacggcgaaaagccgaagctgaccaaatatcattactttctattcgtttttgttctcggagtttcctttatgcttgtcagccagctgttttcttcacctgagaaaactgaaaacgccaaaacgataacggccgtatcatcacaacattccgcagacagcaaagagaaaacagccgaagtgtttaaagcctcaaaatctgataaacctaaagactcgatcgatgactatgaaaaagaatacgaaaatcaactgaaagaaattcttgaaacaatcattggtgtggacgatgtctcggttgtcgtgaatgtagatgcaacgtcgctgaaagtatacgaaaaaaacaaatcaaacaaaaatacaaccactgaagaaactgacaaagaaggcggaaaaagaagcgttacagaccaatcatcggaggaagaaatcgtcatgatcaaaaacggcgataaagaaacacctgtcgtcgtccaaacgaaaaaacctgatatacgcggtgtactcgttgttgctcaaggagtggacaacgttcaaataaaacaaaccattatcgaagcggtgacacgggtc

SEQ ID NO: 22 - DNA, 49 bp, Artificial Sequence
  oligo
gcggccgccatatgcatcctaggccaagcttcccagcgcgcatatattc

SEQ ID NO: 23 - DNA, 50 bp, Artificial Sequence
  oligo
tcattaggcaccccaggctttacacgacccgtgtcaccgcttcgataatg

SEQ ID NO: 24 - DNA, 52 bp, Artificial Sequence
  oligo
catggattcagggagaacctcagcgatttcgctttcttcccttcctttctcg

SEQ ID NO: 25 - DNA, 50 bp, Artificial Sequence
  oligo
cattatcgaagcggtgacacgggtcgtgtaaagcctggggtgcctaatga

SEQ ID NO: 26 - DNA, 55 bp, Artificial Sequence
  oligo
cagaaaaaatcgtggaattgatacactacaatactcacataaggtggtatgttcg

SEQ ID NO: 27 - DNA, 51 bp, Artificial Sequence
  oligo
ggacggaagtccgggttatcggtcgtttacaaaaacagccgctggatatcg

SEQ ID NO: 28 - DNA, 22 bp, Artificial Sequence
  oligo
cagcctcgagggattgaagcgc

SEQ ID NO: 29 - DNA, 51 bp, Artificial Sequence
  oligo
cgatatccagcggctgtttttgtaaacgaccgataacccggacttccgtcc

SEQ ID NO: 30 - DNA, 51 bp, Artificial Sequence
  oligo
gatttatacagaacggccgcggcaaatccggaattcctccattttcttctg

SEQ ID NO: 31 - DNA, 57 bp, Artificial Sequence
  oligo
gataaacgctataactgtattcacgaacataccaccttatgtgagtattatttattg

SEQ ID NO: 32 - DNA, 51 bp, Artificial Sequence
  oligo
cagaagaaaatggaggaattccggatttgccgcggccgttctgtataaatc

SEQ ID NO: 33 - DNA, 57 bp, Artificial Sequence
  oligo
caataaataatactcacataaggtggtatgttcgtgaatacagttatagcgtttatc

SEQ ID NO: 34 - DNA, 57 bp, Artificial Sequence
  oligo
cagaaaaaatcgtggaattgatacactaacaaagagaaactcggaaaggatgaaagc

SEQ ID NO: 35 - DNA, 53 bp, Artificial Sequence
  oligo
ggacggaagtccgggttatcggtcgtacacaatttcctgaggcatcacggtat

SEQ ID NO: 36 - DNA, 57 bp, Artificial Sequence
  oligo
gctttcatcctttccgagtttctctttgttagtgtatcaattccacgattttttctg

SEQ ID NO: 37 - DNA, 53 bp, Artificial Sequence
  oligo
ataccgtgatgcctcaggaaattgtgtacgaccgataacccggacttccgtcc

SEQ ID NO: 38 - protein, 483 aa, Bacillus licheniformis
ANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 39 - protein, 478 aa, Paenibacillus curdlanolyticus
ADNGTIMQYFEWYLPNDGAHWNRLNNDAQNLKNVGITAVWIPPAYKGGSSADVGYGVYDTYDLGEFNQKGTVRTKYGTKSELISAVNNLHAKGIAVYGDVVLNHRMNADATELVDAVEVDPNNRYVETTSTYQIQAWTQYDFPGRGNTYSSWKWRWYHFDGVDWDQSRGLNRIYKLDGKDWDWPVDSEYGNYDYLMGADLDFNHPDVVNETKTWGKWFVNTVNLDGVRLDAVKHIKFDFMRDWVNNVRSTTGKNLFAVGEYWHYDVNKLNSYITKTNGTMSLFDVPLHFRFYDASNGGGGYDMRNLLNNTLMSSNPMKAVTFVENHDTQPGQSLESTVQSWFKPLAYATILTREQGYPCVFYGDYYGTSDGKISSYKPIMDKLLNARKVYAYGTQRDYFDHPDIVGWTREGDAAHAGSGLATLITDGPGGSKWMYVGTSKAGQVWTDKTGNRSGTVTIDANGWGNFWVNKGSVSVWAK

SEQ ID NO: 40 - protein, 425 aa, Bacillus subtilis
LTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPD

SEQ ID NO: 41 - protein, 470 aa, Bacillus subtilis
AGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 42 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGGGSNSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 43 - protein, 269 aa, Bacillus amyloliquefaciens; identical to SEQ ID NO: 42

SEQ ID NO: 44 - DNA, 20 bp, Artificial Sequence
  oligo
cgcctcatcattacggcatc

SEQ ID NO: 45 - DNA, 48 bp, Artificial Sequence
  oligo
cgacctgcaggcatgcaagctaccaccttatgtgagtattgaattgac

SEQ ID NO: 46 - protein, 29 aa, Bacillus subtilis
MRSKKLWISLLFALTLIFTMAFSNMSAQA

(19) United States
(12) Patent Application Publication
(10) Pub. No.: US 2018/0371445 A1
(43) Pub. Date: Dec. 27, 2018

(54) ENHANCED PROTEIN PRODUCTION AND METHODS THEREOF

(71) Applicant: Danisco US Inc., Palo Alto, CA (US)

(72) Inventors: Christina Bongiorni, Palo Alto, CA (US); Jolanda Neef, Palo Alto, CA (US); Brian F. Schmidt, Palo Alto, CA (US); Anita Van Kimmenade, Palo Alto, CA (US); Jan Maarten Van Dijl, Palo Alto, CA (US)

(73) Assignee: Danisco US Inc., Palo Alto, CA (US)

(21) Appl. No.: 16/064,710

(22) PCT Filed: Dec. 21, 2016
(86) PCT No.: PCT/US16/67936

§ 371 Date: Jun. 21, 2018

Related U.S. Application Data
(60) Provisional application No. 62/387,578, filed on Dec. 23, 2015.
(60) Provisional application No. 62/308,440, filed on Mar. 15, 2016.

(51) Int. Cl. C12N 9/54; C12P 21/00

(52) U.S. Cl. CPC C12N 9/54; C12P 21/00; C12Y 304/24

(57) ABSTRACT

The present disclosure is generally related to modified Gram-positive bacterial cells producing increased amounts of one or more protein(s) of interest. Certain embodiments of the instant disclosure are therefore directed to modified Gram-positive bacterial cells expressing an increased amount of a POI relative to unmodified (i.e., parental) Gram-positive bacterial cells, wherein the modified (i.e., daughter) bacterial cells comprise a modification which increases rasP gene expression. In certain other embodiments, the disclosure pertains to methods of modifying bacterial cells such that the modified (daughter) cells produce an increased level of a protein of interest. In other embodiments, the disclosure pertains to a protein of interest produced by fermenting a modified bacterial cell of the instant disclosure. Certain other embodiments of the disclosure are directed to one or more proteinaceous compositions comprising one or more protein(s) of interest thus made.

### CROSS REFERENCE TO RELATED APPLICATIONS

This application claims the benefit of U.S. Provisional Application No. 62/387,578, filed Dec. 23, 2015 and U.S. Provisional Application No. 62/308,440, filed Mar. 16, 2016, each of which are hereby incorporated by reference in their entirety.

### FIELD

The present disclosure is generally related to the fields of bacteriology, microbiology, genetics, molecular biology, enzymology and the like. Certain embodiments are directed to modified Gram-positive bacterial cells expressing and producing increased amounts of one or more protein(s) of interest.

### REFERENCE TO THE SEQUENCE LISTING

The contents of the electronic submission of the text file Sequence Listing, named “NB40883WOPCT_SequenceListing.txt” was created on Nov. 16, 2016 and is 39 KB in size, is hereby incorporated by reference in its entirety.

### BACKGROUND

The production of proteins (e.g., enzymes, antibodies, receptors, etc.) in microbial hosts is of particular interest in the biotechnological arts. Likewise, optimization of microbial host cells for the production and secretion of one or more protein(s) of interest is of high relevance, particularly in the industrial biotechnology setting, wherein small improvements in protein yield are quite significant when the protein is produced in large industrial quantities.

Gram-positive bacteria such as Bacillus subtilis, Bacillus licheniformis and Bacillus amyloliquefaciens are frequently used as microbial factories for the production of industrial relevant proteins, due to their excellent fermentation properties and high yields (up to 25 grams per liter culture; Van Dijl and Hecker, 2013). For example, B. subtilis is well known for its production of α-amylases (Jensen et al., 2000; Raul et al., 2014) and proteases (Brode et al., 1996) necessary for food, textile, laundry, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010).

The Gram-positive bacterium B. subtilis uses various pathways to secrete proteins, of which the Tat-machinery (Raul et al., 2014; Goosens et al., 2014; Tjalsma et al., 2004) and the Sec-machinery (Tjalsma et al., 2004) are the two best studied. The majority of proteins are secreted via the Sec-pathway (Harwood and Cranenburgh, 2007) in an unfolded (non-native) conformation, wherein the hydrophobic signal peptide directs the protein to the translocation machinery of the cell. Shortly after translocation via the SecYEG translocon, promoted by the ATPase SecA, the signal peptide of the protein is liberated (cleaved) by one of the signal peptidases SipS-SipW (Tjalsma et al., 1997) and subsequently degraded by the signal peptide peptidases TepA and SppA (Bolhuis et al., 1999).

In the past 30 years, numerous studies have been performed to improve the secretion of heterologous proteins by B. subtilis, B. licheniformis, B. amyloliquefaciens and the like. The major targets for this optimization have been the composition of the signal peptides, the SRP, the translocation machinery, the signal peptidases and regulatory factors (Kang et al., 2014). Recently it was shown that overexpression of the essential protein PrsA (involved in post-translocational folding) and the chaperone DnaK resulted in improved secretion of amylases in B. subtilis (Chen et al., 2015), demonstrating the impact of proper folding on efficient protein secretion, wherein PrsA was postulated to be the limiting factor for efficient protein secretion (Kontinen and Sarvas, 1993). Furthermore, it was recently demonstrated that PrsA can be degraded by WprA and other proteases located in the cell wall (Krishnappa et al., 2014), rendering it less suitable for industrial use.

Thus, due to the high commercial/industrial use of these microbial cell factories, new targets for the improvement of secretion and/or production of industrially relevant proteins in Gram-positive bacteria are highly desirable in the biotechnological arts. The present disclosure addresses and fulfils the unmet need for increased production and/or secretion of proteins of interest in Gram-positive bacterial cells. More particularly, as set forth below in the Detailed Description, the instant disclosure is directed to the surprising and unexpected findings that Gram-positive bacterial cells modified to express, or over-express, the rasP gene, encoding the “regulating anti-sigma factor protease” or “RasP” (formerly known as YluC), results in increased production of one or more protein(s) of interest from the modified Gram-positive bacterial cells.

### SUMMARY

In certain embodiments the present disclosure is directed to modified Gram-positive bacterial cells producing an increased amount of a protein of interest (hereinafter, a “POI”) relative to an unmodified (parental) Gram-positive bacterial cell, wherein the modified bacterial cell comprises a modification which increases rasP gene expression. In certain embodiments, the modification which increases rasP gene expression is a modification to an endogenous chromosomal rasP gene. In other embodiments, the native promoter of the endogenous chromosomal rasP gene is substituted with any promoter having a higher activity than the native rasP promoter. In certain other embodiments, the native promoter of the endogenous chromosomal rasP gene is substituted with a spoVG promoter or an aprE promoter. In yet other embodiments, the spoVG promoter comprises a nucleotide sequence comprising 95% sequence identity to SEQ ID NO: 3. In other embodiments, the aprE promoter comprises a nucleotide sequence comprising 95% sequence identity to SEQ ID NO: 4.

In certain other embodiments, the modification to an endogenous chromosomal rasP gene is a modification of the native 5′-untranslated region (5′-UTR) of the endogenous chromosomal rasP gene. In other embodiments, the native rasP chromosomal 5′-UTR is replaced with a 5′-UTR comprising 95% sequence identity to the aprE 5′-UTR of SEQ ID NO: 5. In yet other embodiments, the modification to an endogenous chromosomal rasP gene is a modification of both the native promoter and the native 5′-UTR of the endogenous chromosomal rasP gene.

In certain other embodiments, the modification which increases rasP gene expression is an exogenous polynucleotide comprising a rasP gene. In particular embodiments, the exogenous polynucleotide comprising the rasP gene is comprised within an extrachromosomal plasmid. In another embodiment, the extrachromosomal plasmid is an expression cassette. In other embodiments, the extrachromosomal plasmid is an integration plasmid. In certain other embodiments, the plasmid stably integrates into the chromosome of the modified cell.

In other embodiments, the genetic modification increasing rasP expression is a polynucleotide comprising an exogenous rasP open reading frame (ORF), wherein the ORF is operably linked and under the control of a constitutive promoter, an inducible promoter or a conditional promoter. In certain embodiments, the exogenous polynucleotide comprising the rasP ORF is comprised within an extrachromosomal plasmid. In certain embodiments, the extrachromosomal plasmid is an expression cassette. In certain other embodiments, the extrachromosomal plasmid is an integration plasmid. In particular embodiments, plasmid integrates into the chromosome of the modified cell.

In other embodiments, the rasP gene comprises a nucleic acid sequence comprising at least 60% sequence identity to open reading frame (ORF) nucleic acid sequence of SEQ ID NO: 1. In other embodiments, the ORF of SEQ ID NO: 1 encodes a RasP polypeptide, wherein the RasP polypeptide is further defined as a Zn²⁺ metalloprotease having site-2 protease (S2P) activity. In another embodiment, the rasP gene encodes a RasP polypeptide comprising 60% amino acid sequence identity to a RasP polypeptide of SEQ ID NO: 2 and comprises an active site consensus sequence of SEQ ID NO: 6 (LVFFHELGHLL), which aligns with amino acid residues 16 to 26 of the RasP polypeptide of SEQ ID NO: 2. In yet other embodiments, the rasP gene encodes a RasP polypeptide comprising 80% amino acid sequence identity to a RasP polypeptide of SEQ ID NO: 2 and comprises an active site consensus sequence of SEQ ID NO: 7 (HEXXH), which aligns with amino acid residues 20 to 24 of the RasP polypeptide of SEQ ID NO: 2.

In other embodiments, the increased amount of the POI produced, relative to the unmodified (parental) Gram-positive cell, is at least a 5% increase. In another embodiment, the increased amount of the POI produced, relative to the unmodified (parental) Gram-positive cell, is at least a 10% increase.

In certain other embodiments, the Gram-positive bacterial cell is a member of the Bacillus genus. In yet other embodiments, the Bacillus is selected from B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, B. marmaresis, and B. thuringiensis. In another embodiment, the Bacillus is B. subtilis or B. licheniformis.

In certain other embodiments, the POI is encoded by a gene exogenous to the modified bacterial cell or a gene endogenous to the modified bacterial cell. In yet other embodiments, the POI is secreted or transported extracellularly. In further embodiments, the POI secreted or transported extracellularly is further isolated and purified.

In certain other embodiments, the POI is an enzyme. In other embodiments, the enzyme is selected from the group consisting of acetyl esterases, aryl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, thaumatin, transferases, transport proteins, transglutaminases, xylanases, hexose oxidases, and combinations thereof.

In another embodiment, the disclosure is directed to an isolated POI produced by a modified cell of the disclosure.

In certain other embodiments, the disclosure is directed to a method for increasing the production of a POI in a Gram-positive bacterial cell comprising (a) obtaining a modified Gram-positive bacterial cell producing an increased amount of a POI, wherein the modified bacterial cell comprises a modification which increases rasP gene expression, and (b) culturing the modified cell under conditions such that the POI is expressed, wherein the modified bacterial cell producing an increased amount of a POI is relative to the production of the same POI in an unmodified (parental) Gram-positive bacterial cell.

In certain other embodiments, the disclosure is directed to an isolated POI produced by the methods of the instant disclosure.

In other embodiments, the disclosure is directed to a method for obtaining a modified Gram-positive bacterial cell producing an increased amount of a POI comprising (a) introducing into a parental Gram-positive bacterial cell at least one gene modification which increases rasP gene expression, and (b) selecting one or more daughter cells expressing an increased amount of a POI, wherein the one or more daughter cells selected for producing an increased amount of the POI are defined as modified (daughter) Gram-positive bacterial cells.

### BRIEF DESCRIPTION OF DRAWINGS

FIG. 1 compares the expression of AmyE amylase, AmyL amylase and protease BPN′-Y217L secreted into the culture medium from modified B. subtilis cells comprising either the deleted tepA gene (ΔtepA) or the deleted rasP gene (LrasP) relative to unmodified (parental; wild-type) B. subtilis cells. FIG. 1A shows the LDS-PAGE gels of the secreted enzymes (AmyE, AmyL and BPN′-Y217L) from the B. subtilis cells lacking the rasP or tepA genes compared to the B. subtilis cells (parental) cells. FIG. 1B shows the quantification of the secreted AmyE, AmyL and BPN′-Y217L proteins using ImageJ, presented as the ratio of protein secreted from B. subtilis cells comprising a deleted rasP (LrasP) or tepA (ΔtepA) gene relative to the ratio of protein secreted from unmodified (parental) wild-type B. subtilis cells (i.e., ΔtepA cells/wt cells and LrasP/wt cells).

FIG. 2 shows the cell densities measured at OD600 (FIG. 2A) and production (FIG. 2B) of AmyE from wild-type (parental) B. subtilis cells, modified B. subtilis cells comprising and expressing/over-expressing the rasP gene under the control of the spoVG promoter (i.e., the “PspoVG-rasP” expression cassette) and modified B. subtilis cells comprising and expressing/over-expressing the rasP gene under the control of the spoVG promoter and the aprE 5′UTR (i.e., the “PspoVG-UTR-rasP” expression cassette).

FIG. 3 shows shake flask production of amylase PcuAmy1-v6 from wild-type (parental) B. subtilis cells and modified B. subtilis cells comprising and expressing/over-expressing the rasP gene under the control of the spoVG promoter (i.e., the “PspoVG-rasP” expression cassette).

FIG. 4 shows the cell densities (FIG. 4A) and production (FIG. 4B) of amylase PcuAmy1-v6 from wild-type (parental) B. subtilis cells and modified B. subtilis cells comprising and expressing/over-expressing the tepA gene under the control of the spoVG promoter (i.e., the “PspoVG-tepA” expression cassette).

FIG. 5 shows the production of Beta-D-glucanase (BglC) from wild-type (parental) B. subtilis cells and modified B. subtilis cells comprising and expressing/over-expressing the rasP gene under the control of the spoVG promoter (i.e., the “PspoVG-rasP” expression cassette).

FIG. 6 shows the cell densities (FIG. 6A) and production (FIG. 6B) of Properase from wild-type (parental) B. subtilis cells and modified B. subtilis cells comprising and expressing/over-expressing the rasP gene under the control of the spoVG promoter (i.e., the “PspoVG-rasP” expression cassette).

FIG. 7 shows the cell densities (FIG. 7A) and production (FIG. 7B) of the “AmyAc family” α-amylase expressed from wild-type (parental) B. subtilis cells and modified (daughter) B. subtilis comprising and expressing/over-expressing the rasP gene under the control of the spoVG promoter (i.e., the “PspoVG-rasP” expression cassette). The parental and modified (daughter) B. subtilis cells used in this experiment were grown in deep well microtiter plates (DWMTP) using 5SM12 growth medium.

### DETAILED DESCRIPTION

The present disclosure is generally related to modified Gram-positive bacterial cells producing increased amounts of one or more protein(s) of interest (hereinafter, a “POI”). Thus, certain embodiments of the instant disclosure are directed to modified Gram-positive bacterial cells producing an increased amount of a POI relative to unmodified (parental) Gram-positive bacterial cells producing the same POI, wherein the modified bacterial cells comprise a modification which increases rasP gene expression.

As set forth herein, the rasP gene encodes the “regulating anti-sigma factor protease” or “RasP” (formerly known as YluC). RasP belongs to a group of zinc-dependent intramembrane cleaving proteases (or “iClips) and is further defined herein as a site-2 protease (S2P) or site-2 zinc metalloprotease (see, e.g., Saito et al., 2011; Heinrich et al., 2008). In certain embodiments, a RasP polypeptide of the disclosure is further defined as a hydrolase (e.g., a peptidase), and more particularly a metalloendopeptidase, belonging to Enzyme Commission (EC) class 3.4.24 (EC 3.4.24).

In view of the modified bacterial cells, compositions thereof and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference.

It is further noted that the claims may be drafted to exclude any optional element. As such, this statement is intended to serve as antecedent basis for use of such exclusive terminology as “solely,” “only”, “excluding” and the like, in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As defined herein, a “modified cell”, a “modified bacterial cell” or a “modified host cell” may be used interchangeably and refer to recombinant Gram-positive bacterial (host) cells that comprise a modification (e.g., a genetic modification) which increases rasP gene expression. For example, a “modified” Gram-positive bacterial cell of the instant disclosure may be further defined as a “modified (host) cell” which is derived from a parental bacterial cell, wherein the modified (daughter) cell comprises a modification which increases rasP gene expression.

As defined herein, an “unmodified cell”, an “unmodified bacterial cell” or an “unmodified host cell” may be used interchangeably and refer to “unmodified” (parental) Gram-positive bacterial cells that do not comprise a modification which increases rasP gene expression.

As used herein, when the expression and/or production and/or secretion of a POI in an “unmodified” (parental) cell is being compared to the expression and/or production and/or secretion of the same POI in a “modified” (daughter) cell, it will be understood that the “modified” and “unmodified” cells are grown/cultured/fermented under essentially the same conditions (e.g., the same conditions such as media, temperature, pH and the like).

Likewise, as defined herein, the terms “increased production”, “enhanced production”, “increased production of a POI”, “enhanced production of a POI”, and the like refer to a “modified” (daughter) cell comprising a modification which increases rasP gene expression, wherein the “increase” is always relative (vis-à-vis) to an “unmodified” (parental) cell expressing the same POI.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”. Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated region (UTR), and 3′-UTR, as well as the coding sequence.

As used herein, the term “coding sequence” refers to a nucleotide sequence, which directly specifies the amino acid sequence of its (encoded) protein product. The boundaries of the coding sequence are generally determined by an open reading frame, which usually begins with an ATG start codon. The coding sequence typically includes DNA, cDNA, and recombinant nucleotide sequences.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction. For example, in certain embodiments, a modified cell, a modified bacterial cell or a modified host cell that comprises a modification which “increases rasP gene expression” encompasses recombinant Gram-positive bacterial cells that comprise an ORF encoding a RasP polypeptide, wherein the expression of the ORF sequence encoding the RasP polypeptide is operably linked and under the control of a constitutive promoter, an inducible promoter, a conditional promoter, and the like; and optionally comprise 5′ and 3′ regulatory (non-transcribed) nucleic acid sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing sites, effector binding sites, stem-loop structures and the like.

As defined herein, the term “introducing”, as used in phrases such as “introducing into the bacterial cell” at least one polynucleotide open reading frame (ORF), or a gene thereof, or a vector thereof, includes methods known in the art for introducing polynucleotides into a cell, including, but not limited to protoplast fusion, transformation (e.g., calcium chloride, electroporation), transduction, transfection, conjugation and the like (e.g., see Ferrari et al., 1989).

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in the cell that is to be transformed. As used herein, “transformation” refers to the transfer of a nucleic acid molecule into the genome of a host organism, resulting in genetically stable inheritance of the transferred nucleic acid molecule.

As defined herein, a host cell “genome”, a bacterial (host) cell “genome”, or a Gram-positive bacterial (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “transformation cassette” refers to a specific vector comprising a foreign gene (or an ORF thereof), and having elements in addition to the foreign gene that facilitate transformation of a particular host cell.

An “expression cassette” refers to a specific vector comprising a foreign gene (or an ORF thereof), and having elements in addition to the foreign gene that allow for “increased” expression of the foreign (heterologous) gene in a host cell.

Many prokaryotic and eukaryotic expression vectors are commercially available and well known in the art. Selection of appropriate expression vectors is within the knowledge of those having skill in the art.

As used herein, the term “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism.

An “expression vector” refers to a vector that has the ability to incorporate and express heterologous DNA in a foreign cell. Many prokaryotic and eukaryotic expression vectors are commercially available.

A “targeting vector” is a vector that includes polynucleotide sequences that are homologous to a region in the chromosome of a host cell into which it is transformed and that can drive homologous recombination at that region. Targeting vectors find use in introducing mutations or exogenous gene sequences into the chromosome of a cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. Selection and/or construction of appropriate vectors is within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the term “protein of interest” or “POI” refers to a polypeptide of interest that is desired to be expressed in a modified Gram-positive bacterial cell (e.g., a “host” cell), wherein the POI is produced at increased levels (i.e., relative to an unmodified (parental) Gram-positive bacterial cell). Thus, as used herein, a POI may be an enzyme, a substrate-binding protein, a surface-active protein, a structural protein, a receptor protein, an antibody and the like.

Similarly, as defined herein, a “gene of interest” or “GOI” refers to a nucleic acid sequence (e.g., a polynucleotide, a gene or an open reading frame) which encodes a POI. A GOI encoding a POI may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids.

The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. Foreign (heterologous) genes comprise native genes (or ORFs) inserted into a non-native organism and/or chimeric genes inserted into a native or non-native organism.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or an amino acid sequence thereof, the term “substitution” means the replacement (i.e., substitution) of one amino acid with another amino acid.

As defined herein, a “heterologous” nucleic acid construct or a “heterologous” nucleic acid sequence has a portion of the sequence which is not native to the cell in which it is expressed.

As defined herein, a “heterologous control sequence”, refers to a gene expression control sequence (e.g., a promoter or enhancer) which does not function in nature to regulate (control) the expression of the GOI. Generally, heterologous nucleic acid sequences are not endogenous (native) to the cell, or a part of the genome in which they are present, and have been added to the cell, by infection, transfection, transformation, microinjection, electroporation, protoplast fusion and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

As used herein, a “parental” cell refers to any cell or strain of microorganism in which the genome of the “parental” cell is modified (e.g., one or more mutations introduced into the parental cell or one or more genes or ORFs introduced into the parental cell) to generate a modified “daughter” cell.

The term “derived” encompasses the terms “originated” “obtained,” “obtainable,” and “created,” and generally indicates that one specified material or composition finds its origin in another specified material or composition, or has features that can be described with reference to the another specified material or composition.

As used herein, “increasing” protein production is meant an increased amount of protein produced. The protein may be produced inside the host cell, or secreted (or transported) into the culture medium. In certain embodiments, the protein of interest is produced into the culture medium.

Increased protein production may be detected for example, as higher maximal level of protein or enzymatic activity, such as protease activity, amylase activity, cellulase activity, hemicellulase activity and the like, or total extracellular protein produced by the modified (daughter) cell as compared to the unmodified (parental) cell.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wis., USA 53711) (Needleman and Wunsch, (1970).

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

### I. Rasp Polypeptides and Genes Encoding the Same

As set forth previously above, the rasP gene encodes the “regulating anti-sigma factor protease” or “RasP” (formerly known as YluC); which belongs to a group of zinc-dependent intramembrane cleaving proteases (or “iClips) and is further defined herein as a site-2 protease (S2P) or site-2 zinc metalloprotease (see, e.g., Saito et al., 2011; Heinrich et al., 2008). As defined herein, a “rasP” gene (or an ORF thereof) encodes a “RasP” polypeptide, and is not meant to be limited to a specific RasP polypeptide sequence (or rasP gene or ORF sequence encoding the same).

Thus, in certain embodiments, a rasP gene (or ORF thereof) of the instant disclosure may be any nucleic acid (polynucleotide) or any homologue or orthologue nucleic acid sequence thereof encoding a polypeptide characterized as having S2P Zn²⁺ metalloprotease activity, as long as such encoded polypeptide (having S2P Zn²⁺ metalloprotease activity) increases the production of a POI when the rasP nucleic acid or homologue or orthologue nucleic acid sequence thereof is expressed or over-expressed in a modified Gram-positive of the instant disclosure.

For example, the RasP polypeptide isolated from B. subtilis strain 168 (e.g., see, UniProtKB; RASP_BACSU; Accession No. 031754) comprises 422 amino acids (˜46.7 kDa) set forth in SEQ ID NO: 2, wherein histidine amino acid residues at positions 20 and 24 are the putative Zn²⁺ coordination/binding sites and the glutamic acid residue at position 21 is the proteolytic active site.

Thus, in certain embodiments, a gene (or ORF thereof) encoding a RasP polypeptide of the instant disclosure is a gene or ORF encoding a polypeptide comprising about 60% sequence identity to a RasP polypeptide set forth in SEQ ID NO: 2. For example, a BLAST search in the UniProtKB database (using B. subtilis RasP polypeptide sequence of SEQ ID NO: 2 as the subject sequence and the following search parameters: E-threshold=10; Matrix=auto; gapped=yes) identified a range of RasP polypeptides comprising 100% to about 60% sequence identity to SEQ ID NO: 2.

In certain other embodiments, a gene (or ORF thereof) encoding a RasP polypeptide of the instant disclosure is a polypeptide comprising a contiguous active site consensus sequence of SEQ ID NO: 6 (LVFFHELGHLL; wherein the underlined histidine amino acids are the Zn²⁺ binding sites and the bold glutamic acid residue is the active site residue), wherein the amino acid sequence of the RasP polypeptide consensus sequence of SEQ ID NO: 6 can be aligned with the amino acid residues 16 to 26 of the RasP polypeptide of SEQ ID NO: 2 and result in at least 60% sequence homology.

In other embodiments, a gene (or ORF thereof) encoding a RasP polypeptide of the instant disclosure is a polypeptide comprising an active site consensus sequence of SEQ ID NO: 7 (HEXXH; wherein the underlined histidine amino acids are the Zn²⁺ binding sites, the bold glutamic acid residue is the active site residue and X is any amino acid), wherein the amino acid sequence of the RasP polypeptide consensus sequence of SEQ ID NO: 7 can be aligned with the amino acid residues 20 to 24 of the RasP polypeptide of SEQ ID NO: 2 and result in at least 80% sequence homology.

In certain embodiments, a RasP polypeptide of the disclosure is further defined as a metalloendopeptidase belonging to EC class 3.4.24 (EC 3.4.24).

### II. Bacterial Cells and Methods of Use

The present disclosure is generally related to modified Gram-positive bacterial cells producing increased amounts of one or more protein(s) of interest. Thus, certain embodiments the instant disclosure are directed to modified Gram-positive bacterial cells expressing an increased amount of a POI relative to unmodified (i.e., parental) Gram-positive bacterial cells, wherein the modified (i.e., daughter) bacterial cells comprise a modification which increases rasP gene expression.

In certain embodiments, the disclosure pertains to methods of modifying bacterial cells such that the modified (daughter) cells produce an increased level of a protein of interest. In other embodiments, the disclosure pertains to a protein of interest produced by fermenting a modified bacterial cell of the instant disclosure. Certain other embodiments of the disclosure are directed to one or more proteinaceous compositions comprising one or more protein(s) of interest thus made. In yet other embodiments, the present disclosure pertains to methods of producing one or more protein(s) of interest employing modified bacterial cells set forth herein, as well as to methods of producing and using one or more proteinaceous compositions comprising one or more protein(s) of interest.

In certain embodiments, a modification of a bacterial cell which increases rasP gene expression can be any type of genetic modification which enhances or increases the expression of a rasP gene (or ORF thereof) in the modified host. For example, in certain embodiments, a modification of a bacterial cell which increases rasP gene expression comprises codon optimization of the rasP gene (or ORF thereof) for enhanced expression in a desired host cell. In other embodiments, a modification of a host cell which increases rasP gene expression is an expression cassette encoding a RasP polypeptide, wherein the expression cassette is introduced into the (modified) cell. In certain other embodiments, an expression cassette comprising a gene or ORF encoding a RasP polypeptide is under the control of an inducible promoter, a constitutive promoter, a conditional promoter and the like.

In certain embodiments, a promoter for directing the transcription of a polynucleotide sequence encoding a POI or a RasP polypeptide is a wild-type aprE promoter, a mutant aprE promoter or a consensus aprE promoter set forth in PCT International Publication WO2001/51643. In certain other embodiments, a promoter for directing the transcription of a polynucleotide sequence encoding a POI or a RasP polypeptide is a wild-type spoVG promoter, a mutant spoVG promoter or a consensus spoVG promoter (Frisby and Zuber, 1991).

In certain embodiments, a modified bacterial cell of the disclosure is a Bacillaceae family member. In other embodiments, a modified bacterial cell of the disclosure is a member of the Bacillus genus. In certain embodiments, a modified bacterial cell of the disclosure is a Bacillus cell selected from B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii and B. thuringiensis, B. marmarensis. In other embodiments, the Bacillus cell is Bacillus subtilis or Bacillus licheniformis.

It is recognized in the art that the genus Bacillus continues to undergo taxonomical reorganization. Thus, it is intended that the genus include species that have been reclassified, including, but not limited to, such organisms as B. stearothermophilus, which is now named “Geobacillus stearothermophilus”, or B. polymyxa, which is now “Paenibacillus polymyxa”.

### III. Modified Cells Producing an Increased Level of a Protein of Interest

To confirm that a modified Gram-positive bacterial cell produces an increased level of a POI, various methods of screening can be applied. For instance, an expression vector may encode a polypeptide fusion to the target protein and serves as a detectable label, or alternatively, the target protein itself may serve as the selectable or screenable marker. The labeled protein can also be detected using Western blotting, dot blotting (detailed descriptions of such methods are available at the website of the Cold Spring Harbor Protocols), ELISA, or, if the label is a GFP, whole cell fluorescence or FACS.

For example, a 6-histidine tag can be included to make a fusion to the target protein, and Western blots can be used to detect such a tag. Moreover, if the target protein expresses at sufficiently high levels, SDS-PAGE combined with Coomassie/silver staining, may be performed to adequately detect increases in mutant expression over wild type; and in such a case, no labeling of any molecules would be necessary.

In other embodiments, the expression of the POI in a modified (host) cell versus an unmodified (parental) cell is correlated with mRNA transcript levels. For example, certain embodiments are related to the molecular characterization of a gene or ORF encoding a POI, which usually includes a thorough analysis of the temporal and spatial distribution of RNA expression. A number of widely used procedures exist and are known in the art for detecting and determining the abundance of a particular mRNA in a total or poly(A) RNA sample. Non-limiting examples include such methods as Northern blot analysis, nuclease protection assays (NPA), in situ hybridization, and reverse transcription-polymerase chain reaction (RT-PCR).

Other methods can be employed to confirm the improved level of a protein of interest, including, for example, the detection of the increase of protein activity or amount per cell, protein activity or amount per milliliter of medium, allowing cultures or fermentations to continue efficiently for longer periods of time, or through a combination of these methods.

The detection of specific productivity is another suitable method for evaluating protein production. Specific productivity (Qp) can be determined using the following equation:

Qp=gP/gDCW·hr

herein, “gP” is grams of protein produced in the tank; “gDCW” is grams of dry cell weight (DCW) in the tank and “hr” is fermentation time in hours from the time of inoculation, which includes the time of production as well as growth time.

In certain embodiments, a modified bacterial cell of the disclosure produces at least about 5%, at least about 6%, at least about 7%, at least about 8%, at least about 9%, or at least about 10% or more of a POI, as compared to its unmodified (parental) cell.

### IV. Culturing Modified Cells for Production of a Protein of Interest

In other embodiments, the present disclosure provides methods for increasing the protein productivity of a modified bacterial cell, as compared (i.e., relative) to an unmodified (parental) cell. More particularly, in certain embodiments, methods for increasing the protein productivity of a modified bacterial cell comprises culturing the modified bacterial cells under suitable fermentation conditions, wherein the modified cell comprises a modification which increases rasP gene expression.

Thus, host cells and transformed cells can be cultured in conventional nutrient media. The culture media for transformed host cells may be modified as appropriate for activating promoters and selecting transformants. The specific culture conditions, such as temperature, pH and the like, may be those that are used for the host cell selected for expression, and will be apparent to those skilled in the art. In addition, culture conditions may be found in the scientific literature such as Sambrook (1982; 2001), Harwood et al. (1990) and from the American Type Culture Collection (ATCC).

Thus, in certain embodiments, the instant disclosure is directed to methods of producing a POI comprising fermenting a modified bacterial cell, wherein the modified cell secrets the POI into the culture medium. Fermentation methods well known in the art can be applied to ferment the modified and unmodified bacterial cells.

In some embodiments, the bacterial cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

This, in certain embodiments, a POI produced by a transformed (modified) host cell may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. Transformation

A polynucleotide construct comprising a nucleic acid encoding a RasP polypeptide or a POI of the disclosure can be constructed such that it is expressed by a host cell. Because of the known degeneracies in the genetic code, different polynucleotides encoding an identical amino acid sequence can be designed and made with routine skills in the art. For example, codon optimizations can be applied to optimize production in a particular host cell.

Nucleic acids encoding proteins of interest can be incorporated into a vector, wherein the vector can be transferred into a host cell using well-known transformation techniques, such as those disclosed herein.

The vector may be any vector that can be transformed into and replicated within a host cell. For example, a vector comprising a nucleic acid encoding a POI can be transformed and replicated in a bacterial host cell as a means of propagating and amplifying the vector. The vector also may be transformed into an expression host, so that the protein encoding nucleic acids (i.e., ORFs) can be expressed as a functional protein. Bacterial cells that serve as expression hosts (i.e, a modified bacterial “host” cell) include members of the Bacillaceae family and members of the Bacillus genus.

A representative vector which can be modified with routine skill to comprise and express a nucleic acid encoding a POI is vector p2JM103BBI (see, Vogtentanz, 2007).

As stated briefly above, a polynucleotide encoding a RasP polypeptide or a POI can be operably linked to a suitable promoter, which allows transcription in the host cell. The promoter may be any nucleic acid sequence that shows transcriptional activity in the host cell of choice and may be derived from genes encoding proteins either homologous or heterologous to the host cell. For example, in certain embodiments, a modification which increases the expression of a rasP gene comprises substituting the native rasP gene promoter with any promoter having a higher activity than the native rasP promoter. Means of assessing promoter activity/strength are routine for the skilled artisan.

Examples of suitable promoters for directing the transcription of a polynucleotide sequence encoding RasP polypeptide or a POI of the disclosure, especially in a bacterial host, include the promoter of the lac operon of E. coli, the Streptomyces coelicolor agarase gene dagA or celA promoters, the promoters of the Bacillus licheniformis alpha-amylase gene (amyL), the promoters of the Bacillus stearothermophilus maltogenic amylase gene (amyM), the promoters of the Bacillus amyloliquefaciens alpha-amylase (amyQ), the promoters of the Bacillus subtilis xylA and xylB genes, and the like.

In certain embodiments, a promoter for directing the transcription of a polynucleotide sequence encoding a POI or a RasP polypeptide is a wild-type aprE promoter, a mutant aprE promoter or a consensus aprE promoter set forth in PCT International Publication WO2001/51643. In certain other embodiments, a promoter for directing the transcription of a polynucleotide sequence encoding a POI or a RasP polypeptide is a wild-type spoVG promoter, a mutant spoVG promoter or a consensus spoVG promoter (Frisby and Zuber, 1991).

In certain embodiments, an aprE promoter comprises a nucleic acid sequence comprising about 90-95% sequence identity to SEQ ID NO: 4.

In other embodiments, a spoVG promoter comprises a nucleic acid sequence comprising about 90-95% sequence identity SEQ ID NO: 3.

In other embodiments, a promoter for directing the transcription of the polynucleotide sequence encoding RasP polypeptide or a POI is a ribosomal promoter such as a ribosomal RNA promoter or a ribosomal protein promoter. More particularly, in certain embodiments, the ribosomal RNA promoter is a rrn promoter derived from B. subtilis, more particularly, the rrn promoter is a rrnB, rrnI or rrnE ribosomal promoter from B. subtilis. In certain embodiments, the ribosomal RNA promoter is a P2 rrnI promoter from B. subtilis set forth in PCT International Publication No. WO2013/086219.

The RasP or POI coding sequence can be operably linked to a signal sequence. The nucleic acid sequence encoding the signal sequence may be the DNA sequence naturally associated with the rasP gene or the GOI (encoding the POI) to be expressed, or may be from a different genus or species. A signal sequence and a promoter sequence comprising a polynucleotide construct or vector can be introduced into a bacterial host cell, and those sequences may be derived from the same source or different sources. For example, in certain embodiments, the signal sequence is an aprE signal sequence (see, e.g., Vogtentanz et al., 2007; Wang et al., 1988) that is operably linked to an aprE promoter set forth in PCT International Publication WO2001/51643.

In certain embodiments, a modification to an endogenous chromosomal rasP gene is a modification of the native 5′-untranslated region (5′-UTR) of the endogenous chromosomal rasP gene. In another embodiment, native rasP chromosomal 5′-UTR is replaced with a 5′-UTR comprising about 90-95% sequence identity to the aprE 5′-UTR of SEQ ID NO: 5.

An expression vector may also comprise a suitable transcription terminator and, in eukaryotes, certain polyadenylation sequences operably linked to the DNA sequence encoding the protein of interest. Termination and polyadenylation sequences may suitably be derived from the same sources as the promoter, but in other embodiments, the termination and polyadenylation sequences may well be derived from different sources as each other and/or as the promoter.

A suitable vector may further comprise a nucleic acid sequence enabling the vector to replicate in the host cell. Examples of such enabling sequences include the origins of replication of plasmids pUC19, pACYC177, pUB110, pE194, pAMB1, pIJ702, and the like.

A suitable vector may also comprise a selectable marker, e.g., a gene the product of which complements a defect in the isolated host cell, such as the dal genes from B. subtilis or B. licheniformis; or a gene that confers antibiotic resistance such as, e.g., ampicillin resistance, kanamycin resistance, chloramphenicol resistance, tetracycline resistance and the like.

A suitable expression vector typically includes components of a cloning vector, such as, for example, an element that permits autonomous replication of the vector in the selected host organism and one or more phenotypically detectable markers for selection purposes. Expression vectors typically also comprise control nucleotide sequences such as, for example, promoter, operator, ribosome binding site, translation initiation signal and optionally, a repressor gene, one or more activator genes sequences, or the like.

Additionally, a suitable expression vector may further comprise a sequence coding for an amino acid sequence capable of targeting the protein of interest to a host cell organelle such as a peroxisome, or to a particular host cell compartment. Such a targeting sequence may be, for example, the amino acid sequence “SKL”. For expression under the direction of control sequences, the nucleic acid sequence of the protein of interest can be operably linked to the control sequences in a suitable manner such that the expression takes place.

Protocols, such as described herein, used to ligate the DNA construct encoding a protein of interest, promoters, terminators and/or other elements, and to insert them into suitable vectors containing the information necessary for replication, are well known to persons skilled in the art (see, e.g., Sambrook et al., 1989, and 3rd edition 2001).

An isolated cell, either comprising a polynucleotide construct or an expression vector, is advantageously used as a host cell in the recombinant production of a POI. The cell may be transformed with the DNA construct encoding the POI, conveniently by integrating the construct (in one or more copies) into the host chromosome. Integration is generally deemed an advantage, as the DNA sequence thus introduced is more likely to be stably maintained in the cell. Integration of the DNA constructs into the host chromosome may be performed applying conventional methods, for example, by homologous or heterologous recombination. Alternatively, the cell may be transformed with an expression vector as described above in connection with the different types of host cells.

It is, in other embodiments, advantageous to delete genes from expression hosts, where the gene deficiency can be cured by an expression vector. Known methods may be used to obtain a bacterial host cell having one or more inactivated genes. Gene inactivation may be accomplished by complete or partial deletion, by insertional inactivation or by any other means that renders a gene nonfunctional for its intended purpose, such that the gene is prevented from expression of a functional protein.

Techniques for transformation of bacteria and culturing the bacteria are standard and well known in the art. They can be used to transform the improved hosts of the present disclosure for the production of recombinant proteins of interest. Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; gene gun or biolistic transformation and protoplast fusion, and the like. Transformation and expression methods for bacteria are also disclosed in Brigidi et al. (1990). A preferred general transformation and expression protocol for protease deleted Bacillus strains is provided in Ferrari et al., U.S. Pat. No. 5,264,366.

### VI. Proteins of Interest Produced by Modified Cells

In another embodiment, the present disclosure provides methods for producing increased levels of a POI comprising obtaining a modified Gram-positive bacterial cell expressing an increased amount of a POI, wherein the modified bacterial cell comprises modification which increase expression of a rasP gene (or ORF thereof), and culturing the modified cell under conditions such that the POI is expressed, wherein the modified bacterial cell expressing an increased amount of a POI is relative to the expression of the POI in an unmodified Gram-positive bacterial cell.

The POI can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

In certain embodiments, a POI or a variant POI thereof is selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

The POI or variant POI may also be a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), and variants thereof or fragments thereof.

Other types of proteins (or variants) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

### VII. Use of the Proteinaceous Compositions

In another embodiment, the present disclosure provides a proteinaceous composition comprising one or more protein(s) of interest. The proteinaceous composition is suitably produced using the methods provided herein. The proteinaceous composition comprises a protein of interest, encoded by a gene of interest, expressed using a method described herein. The composition may be used in various useful industrial applications such as, for example, in biomass hydrolysis, cleaning applications, grain processing, animal nutrition, food composition, textile treatment, personal care products and the like.

For example, a proteinaceous composition thus produced can be used in cleaning application. Enzymatic cleaning components are popular because of their ability to break down soils, stains, and other debris that are otherwise not readily removed by conventional chemical detergents. Well-known enzymes useful for cleaning include proteases and amylases, with other enzymes such as lipases, pectinases, mannanases, even certain cellulases, each providing a set of different functionalities. Proteases combat protein-based stains; amylases work on carbohydrates and starches; and lipases break down lipids or fats, for example. The disclosure provides modified bacterial cells, which have demonstrated improved protein production, suitable and advantageous as a producer of industrial enzymes, variants, and mixtures of interest to such use in cleaning applications.

In another example, the proteinaceous composition thus made can be used in grain procession. Starch is the most common storage carbohydrate in plants, used by the plants themselves as well as by microbes and by higher organisms. A great variety of enzymes are able to catalyze starch hydrolysis. Starch from all plant sources occurs in the form of granules, but depending on the species of the plant source, starch presents in markedly different size and physical characteristics. Acid hydrolysis of starch had widespread use in the past, however this process has now largely been replaced by enzymatic processes, which are known to demand less corrosion-resistant materials and other benefits, need less energy for heating and are relatively easier to control than the acid process. The disclosure provides an engineered, transformed, or derived eubacterial cell, which has demonstrated improved protein production, suitable and advantageous as a producer of industrial enzymes, variants, and mixtures of interest to such use in starch degradation and grain processing.

In another example, the proteinaceous composition thus made can be used in food application. Enzymes produced by bacteria, yeasts and moulds have been used in food application to make foods such as bread, cheese, beer and wine for many thousands of years. Today enzymes are used in bakery, cheese making, starch processing and production of fruit juices and other drinks, providing various benefits such improved texture, appearance and nutritional value, generate desirable flavors and aromas, and the like. Food enzymes typically originate in animals and plants (for example, a starch-digesting enzyme, amylase, can be obtained from germinating barley seeds) as well as from a range of beneficial microorganisms. Enzymes are deemed viable and desirable alternatives to traditional chemical-based technology, replacing synthetic chemicals in many processes. Enzymes can help improve the environmental performance of food production processes, reducing energy consumption and improving biodegradability of waste or side products. Enzymes tend to be more specific in their actions than synthetic chemicals, and as such, enzymatic processes tend to give fewer side reactions and waste or byproducts, and consequently producing higher quality products and reducing the likelihood of pollution. Enzymatic processes are often also the only processes possible. An example of this is in the production of clear apple juice concentrate, which relies on the use of the enzyme pectinase. Most of the food enzymes are produced from microorganisms such Bacillus, Aspergillus, Streptomyces or Kluyveromyces. The disclosure provides an engineered, transformed, or derived eubacterial cell, which has demonstrated improved protein production, suitable and advantageous as a producer of industrial enzymes, variants, and mixtures of interest to such use in food applications.

In another example, the proteinaceous composition thus made can be used in animal feed additive. Cellulases, xylanases, β-glucanases, proteases, lipases, phytases and other carbohydrase of interest have been widely used in animal feed industry. Since many plant based feeds contain substances with anti-nutritional factors that reduce animal growth, the enzymes added to such feeds improve digestibility of these anti-nutritional factors by degrading fibres, proteins, starches and phytates, rendering them more digestible by the animals, and enabling the use of cheaper and often locally produced feeds, while maximizing meat, egg or milk productivity. At the same time, the enzymes added to such feeds also may provide benefits supporting gut health and enhanced animal performance. The disclosure provides an engineered, transformed, or derived eubacterial cell, which has demonstrated improved protein production, suitable and advantageous as a producer of industrial enzymes, variants, and mixtures of interest to such use in animal feed applications.

In yet a further example, the proteinaceous composition thus made can be used in textile applications. Enzymes have become an integral part of the textile processing. There are two well-established enzyme applications in the textile industry. First, enzymes such as amylases are commonly used in the preparatory finishing area for desizing. Second, enzymes such as cellulases are commonly used in the finishing area for softening, bio-stoning and reducing of pilling propensity of cotton goods.

Other enzymes such as, for example, pectinases, lipases, proteases, catalases, xylanases etc., are also used in textile processing. Moreover, there are various applications which entail enzymes included fading of denim and non-denim, bio-scouring, bio-polishing, wool finishing, peroxide removal, decolourization of dyestuff, etc. Thus, in certain embodiments the disclosure provides modified Gram-positive bacterial cells (which are demonstrated herein as having improved protein production) as a producer of industrial enzymes, variants, and mixtures of interest to such use in textiles applications.

Non-limiting examples of compositions and methods disclosed herein are as follows:

1. A modified Gram-positive bacterial cell producing an increased amount of a protein of interest (POI) relative to an unmodified (parental) Gram-positive bacterial cell, wherein the modified bacterial cell comprises a modification which increases rasP gene expression.

2. The modified cell of claim 1, wherein the modification which increases rasP gene expression is a modification to an endogenous chromosomal rasP gene.

3. The modified cell of claim 2, wherein the native promoter of the endogenous chromosomal rasP gene is substituted with any promoter having a higher activity than the native rasP promoter.

4. The modified cell of claim 2, wherein the native promoter of the endogenous chromosomal rasP gene is substituted with a spoVG promoter or an aprE promoter.

5. The modified cell of claim 4, wherein the spoVG promoter comprises a nucleotide sequence comprising 95% sequence identity to SEQ ID NO: 3.

6. The modified cell of claim 4, wherein the aprE promoter comprises a nucleotide sequence comprising 95% sequence identity to SEQ ID NO: 4.

7. The modified cell of claim 2, wherein the modification to an endogenous chromosomal rasP gene is a modification of the native 5′-untranslated region (5′-UTR) of the endogenous chromosomal rasP gene.

8. The modified cell of claim 7, wherein the native rasP chromosomal 5′-UTR is replaced with a 5′-UTR comprising 95% sequence identity to the aprE 5′-UTR of SEQ ID NO: 5.

9. The modified cell of claim 2, wherein the modification to an endogenous chromosomal rasP gene is a modification of both the native promoter and the native 5′-UTR of the endogenous chromosomal rasP gene.

10. The modified cell of claim 1, wherein the modification which increases rasP gene expression is an exogenous polynucleotide comprising a rasP gene.

11. The modified cell of claim 10, wherein exogenous polynucleotide comprising the rasP gene is comprised within an extrachromosomal plasmid.

12. The modified cell of claim 11, wherein the extrachromosomal plasmid is an expression cassette.

13. The modified cell of claim 11, wherein the extrachromosomal plasmid is an integration plasmid.

14. The modified cell of claim 13, wherein the plasmid stably integrates into the chromosome of the modified cell.

15. The modified cell of claim 1, wherein the genetic modification increasing rasP expression is a polynucleotide comprising an exogenous rasP open reading frame (ORF), wherein the ORF is operably linked and under the control of a constitutive promoter, an inducible promoter or a conditional promoter.

16. The modified cell of claim 15, wherein exogenous polynucleotide comprising the rasP ORF is comprised within an extrachromosomal plasmid.

17. The modified cell of claim 16, wherein the extrachromosomal plasmid is an expression cassette.

18. The modified cell of claim 16, wherein the extrachromosomal plasmid is an integration plasmid.

19. The modified cell of claim 18, wherein the plasmid integrates into the chromosome of the modified cell.

20. The modified cell of claim 1, wherein the rasP gene comprises a nucleic acid sequence comprising at least 60% sequence identity to open reading frame (ORF) nucleic acid sequence of SEQ ID NO: 1.

21. The modified cell of claim 20, wherein the ORF of SEQ ID NO: 1 encodes a RasP polypeptide, wherein the RasP polypeptide is further defined as a Zn²⁺ metalloprotease having site-2 protease (S2P) activity.

22. The modified cell of claim 1, wherein the rasP gene encodes a RasP polypeptide comprising 60% amino acid sequence identity to a RasP polypeptide of SEQ ID NO: 2 and comprises an active site consensus sequence of SEQ ID NO: 6, which aligns with amino acid residues 16 to 26 of the RasP polypeptide of SEQ ID NO: 2.

23. The modified cell of claim 1, wherein the rasP gene encodes a RasP polypeptide comprising 80% amino acid sequence identity to a RasP polypeptide of SEQ ID NO: 2 and comprises an active site consensus sequence of SEQ ID NO: 7 (HEXXH), which aligns with amino acid residues 20 to 24 of the RasP polypeptide of SEQ ID NO: 2.

24. The modified cell of claim 1, wherein the increased amount of the POI produced, relative to the unmodified (parental) Gram-positive cell, is at least a 5% increase.

25. The modified cell of claim 1, wherein the increased amount of the POI produced, relative to the unmodified (parental) Gram-positive cell, is at least a 10% increase.

26. The modified cell of claim 1, wherein the Gram-positive bacterial cell is a member of the Bacillus genus.

27. The modified cell of claim 26, wherein the Bacillus is selected from B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, B. marmarensis and B. thuringiensis.

28. The modified cell of claim 27, wherein the Bacillus is B. subtilis or B. licheniformis.

29. The modified cell of claim 1, wherein the POI is encoded by a gene exogenous to the modified bacterial cell or a gene endogenous to the modified bacterial cell.

30. The modified cell of claim 1, wherein the POI is secreted or transported extracellularly.

31. The modified cell of claim 30, wherein the POI secreted or transported extracellularly is further isolated and purified.

32. The modified cell of claim 1, wherein the POI is an enzyme.

33. The modified cell of claim 32, wherein the enzyme is selected from the group consisting of acetyl esterases, aryl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, thaumatin, transferases, transport proteins, transglutaminases, xylanases, hexose oxidases, and combinations thereof.

34. An isolated POI produced by the modified cell of claim 1.

35. A method for increasing the production of a POI in a Gram-positive bacterial cell comprising:
(a) obtaining a modified Gram-positive bacterial cell producing an increased amount of a POI, wherein the modified bacterial cell comprises a modification which increases rasP gene expression, and
(b) culturing the modified cell under conditions such that the POI is expressed, wherein the modified bacterial cell producing an increased amount of a POI is relative to the production of the same POI in an unmodified (parental) Gram-positive bacterial cell.

36. The method of claim 35, wherein the modification which increases rasP gene expression is a modification to an endogenous chromosomal rasP gene.

37. The method of claim 36, wherein the native promoter of the endogenous chromosomal rasP gene is substituted with any promoter having a higher activity than the native rasP promoter.

38. The method of claim 35, wherein the native promoter of the endogenous chromosomal rasP gene is substituted with a spoVG promoter or an aprE promoter.

39. The method of claim 38, wherein the spoVG promoter comprises a nucleotide sequence comprising 95% sequence identity to SEQ ID NO: 3.

40. The method of claim 38, wherein the aprE promoter comprises a nucleotide sequence comprising 95% sequence identity to SEQ ID NO: 4.

41. The method of claim 35, wherein the modification to an endogenous chromosomal rasP gene is a modification of the native 5′-untranslated region (5′-UTR) of the endogenous chromosomal rasP gene.

42. The method of claim 41, wherein the native rasP chromosomal 5′-UTR is replaced with a 5′-UTR comprising 95% sequence identity to the aprE 5′-UTR of SEQ ID NO: 5.

43. The method of claim 35, wherein the modification to an endogenous chromosomal rasP gene is a modification of both the native promoter and the native 5′-UTR of the endogenous chromosomal rasP gene.

44. The method of claim 35, wherein the modification which increases rasP gene expression is an exogenous polynucleotide comprising a rasP gene.

45. The method of claim 44, wherein exogenous polynucleotide comprising the rasP gene is comprised within an extrachromosomal plasmid.

46. The method of claim 45, wherein the extrachromosomal plasmid is an expression cassette.

47. The method of claim 45, wherein the extrachromosomal plasmid is an integration plasmid.

48. The method of claim 47, wherein the plasmid integrates into the chromosome of the modified cell.

49. The method of claim 35, wherein the genetic modification increasing rasP expression is a polynucleotide comprising an exogenous rasP open reading frame (ORF), wherein the ORF is operably linked and under the control of a constitutive promoter, an inducible promoter or a conditional promoter.

50. The method of claim 49, wherein the exogenous polynucleotide comprising the rasP ORF is comprised within an extrachromosomal plasmid.

51. The method of claim 50, wherein the extrachromosomal plasmid is an expression cassette.

52. The method of claim 50, wherein the extrachromosomal plasmid is an integration plasmid.

53. The method of claim 52, wherein the plasmid integrates into the chromosome of the modified cell.

54. The method of claim 35, wherein the rasP gene comprises a nucleic acid sequence comprising at least 60% sequence identity to open reading frame (ORF) nucleic acid sequence of SEQ ID NO: 1.

55. The method of claim 54, wherein the ORF of SEQ ID NO: 1 encodes a RasP polypeptide, wherein the RasP polypeptide is further defined as a Zn²⁺ metalloprotease having site-2 protease (S2P) activity.

56. The method of claim 35, wherein the rasP gene encodes a RasP polypeptide comprising 60% amino acid sequence identity to a RasP polypeptide of SEQ ID NO: 2 and comprises an active site consensus sequence of SEQ ID NO: 7, which aligns with amino acid residues 16 to 26 of the RasP polypeptide of SEQ ID NO: 2.

57. The method of claim 35, wherein the rasP gene encodes a RasP polypeptide comprising 80% amino acid sequence identity to a RasP polypeptide of SEQ ID NO: 2 and comprises an active site consensus sequence of SEQ ID NO: 7, which aligns with amino acid residues 20 to 24 of the RasP polypeptide of SEQ ID NO: 2.

58. The method of claim 35, wherein the increased amount of a POI produced, relative to the unmodified (parental) Gram-positive cell, is at least a 5% increase.

59. The method of claim 35, wherein the increased amount of a POI produced, relative to the unmodified (parental) Gram-positive cell, is at least a 10% increase.

60. The method of claim 35, wherein the Gram-positive bacterial cell is a member of the Bacillus genus.

61. The method of claim 60, wherein the Bacillus is selected from B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, B. marmarensis and B. thuringiensis.

62. The method of claim 61, wherein the Bacillus is B. subtilis or B. licheniformis.

63. The method of claim 35, wherein the POI is encoded by a gene exogenous to the modified bacterial cell or a gene endogenous to the modified bacterial cell.

64. The method of claim 35, wherein the POI is secreted or transported extracellularly.

65. The method of claim 64, wherein the POI secreted or transported extracellularly is further isolated and purified.

66. The method of claim 35, wherein the POI is an enzyme.

67. The method of claim 66, wherein the enzyme is selected from the group consisting of acetyl esterases, aryl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, thaumatin, transferases, transport proteins, transglutaminases, xylanases, hexose oxidases, and combinations thereof.

68. An isolated POI produced by the method of claim 35.

69. A method for obtaining a modified Gram-positive bacterial cell producing an increased amount of a POI comprising:
(a) introducing into a parental Gram-positive bacterial cell at least one gene modification which increases rasP gene expression, and
(b) selecting one or more daughter cells expressing an increased amount of a POI, wherein the one or more daughter cells selected for producing an increased amount of the POI are defined as modified (daughter) Gram-positive bacterial cells.

### EXAMPLES

Certain aspects of the present disclosure may be further understood in light of the following examples, which should not be construed as limiting. Modifications to materials and methods will be apparent to those skilled in the art.

### Example 1

### Construction of Modified Bacillus Subtilis Host Cells

Taq polymerase, dNTPs and buffers were purchased from Takara Bio Inc. (Clontech Laboratories, Inc.; Mountain View, Calif.) and used for the construction of the mutant B. subtilis cells described below. Phusion High Fidelity DNA polymerase (New England Biolabs; Ipswich, Mass.) was used for the construction of the vectors. Primers were obtained from Eurogentec (Liege, Belgium).

A. Construction of a Modified B. subtilis Host Comprising a Deleted rasP Gene.

The construction of a rasP deletion mutant (“ΔrasP”) in a B. subtilis cells was performed using the modified mutation delivery method described by Fabret et al. (2002), wherein the parental B. subtilis cells comprise a deleted upp gene (“Δupp”), as set forth in Fabret et al. (2002). To completely replace (delete) the rasP gene in the B. subtilis (Δupp) cells, the 5′ and 3′ flanking regions of the rasP gene were amplified using PCR primer pairs of SEQ ID NO: 44 and SEQ ID NO: 45.

The amplified rasP fragments were fused to a phleomycin resistance cassette comprising the upp gene and the cl gene (see, Fabret et al., 2002). The resulting fusion product was used to transform competent B. subtilis Δupp::neoR (parental) cells, where competence was induced with 0.3% xylose. This resulted in phleomycin resistant and neomycin sensitive (daughter) cells lacking the target rasP gene. The PCR reactions were performed using the oligonucleotide pairs of SEQ ID NO: 10/SEQ ID NO: 9 and oligonucleotide primers of SEQ ID NO: 10/SEQ ID NO: 11 to verify the correct deletion of the target rasP gene.

B. Construction of a Modified B. subtilis Host Comprising a Deleted tepA Gene

The modified mutation delivery method of Fabret et al. (2002) was further utilized to construct a deletion mutant of the tepA gene (“ΔtepA”) in the B. subtilis (Δupp) cells. Thus, to completely replace the tepA gene, the 5′ and 3′ flanking regions of these genes were amplified using the PCR primer pairs SEQ ID NO: 12/SEQ ID NO: 13 and SEQ ID NO: 14/SEQ ID NO: 15. The amplified fragments were then fused to a phleomycin resistance cassette containing the upp and cl genes (see, Fabret et al., 2002). The resulting fusion product was then used to transform competent B. subtilis Δupp::neoR (parental) cells, where competence was induced with 0.3% xylose. This resulted in phleomycin resistant and neomycin sensitive strains lacking the target tepA gene. PCR reactions were performed to verify the correct deletion of the tepA gene using primer combinations of SEQ ID NO: 16/SEQ ID NO: 15 and SEQ ID NO: 16/SEQ ID NO: 11.

C. Construction of Modified B. Subtilis Cells Over-Expressing the rasP Gene

1. Construction of the Integration Vector pRS-spoIIIAA˜AG.

A 1,040 base pair DNA fragment (SEQ ID NO: 17) of the spoIIIAA˜AB genes was amplified using the primer pair of SEQ ID NO: 18/SEQ ID NO: 19. A spectinomycin resistant marker, flanked by two lox sequences (i.e., SEQ ID NO: 20), was synthetically ordered as g-Block from IDT (Integrated DNA Technologies). A 981 base pair DNA sequence in the spoIIIAF˜AG genes (i.e., SEQ ID NO: 21) was amplified using the oligonucleotides of SEQ ID NO: 22 and SEQ ID NO: 23. The commercially available plasmid “pRS426” was amplified using the oligonucleotides of SEQ ID NO: 24 and SEQ ID NO: 25. Subsequently, the above DNA sequences (i.e., SEQ ID NO: 17, SEQ ID NO: 20, SEQ ID NO: 21) and the pRS426 amplified vector were assembled in vitro using a Gibson Assembly® HiFi 1 Step Kit (SGI) to generate the integration vector “pRS-spoIIIAA˜AG”.

2. Construction of the Plasmid pRS-spoIIIAA˜AG-PspoVG-rasP.

For certain rasP overexpression experiments described herein, the promoter of the B. subtilis spoVG gene was used to drive (over-express) the rasP gene. For example, a synthetic g-Block containing the sequence of the spoVG promoter (SEQ ID NO: 3) was ordered from IDT (Integrated DNA Technologies). The rasP coding sequence was amplified by PCR using the oligonucleotides of SEQ ID NO: 26 and SEQ ID NO: 27. The plasmid designated as “pRS-spoIIIAA˜AG” was amplified using the oligonucleotides of SEQ ID NO: 28 and SEQ ID NO: 29. The plasmid “pRS-spoIIIAA˜AG-PspoVG-rasP” was made by assembly of the spoVG promoter sequence, the rasP coding sequence and the plasmid pRS-spoIIIAA˜AG, using a Gibson Assembly® HiFi 1 Step Kit (SGI). To further increase the expression and production of the RasP polypeptide, the aprE (gene) leader sequence (i.e., 5′-UTR) of SEQ ID NO: 5 was cloned in front (5′) of the rasP gene, to generate the plasmid “pRS-spoIIIAA˜AG-PspoVG-UTR-rasP”.

3. Construction of the Plasmid pRS-spoIIIAA˜AG-PaprE-rasP.

For constructing the rasP (gene) over-expression plasmid termed herein as “pRS-spoIIIAA˜AG-PaprE-rasP”, the B. subtilis aprE (gene) promoter (“PaprE”; SEQ ID NO: 4) was incorporated into the vector, 5′ and operably linked to the rasP coding sequence. The nucleotide sequence of the B. subtilis aprE promoter was amplified by PCR from the genome of B. subtilis using the oligonucleotides of SEQ ID NO: 30 and SEQ ID NO: 31. The plasmid “pRS-spoIIIAA˜AG-PspoVG-rasP” was amplified by PCR using the oligonucleotides of SEQ ID NO: 32 and SEQ ID NO: 33. The aprE promoter (PaprE) and plasmid were ligated with a Gibson Assembly® HiFi 1 Step Kit (SGI).

4. Construction of the Plasmid pRS-spoIIIAA˜AG-PspoVG-tepA.

The nucleotide sequence of the signal peptide peptidase tepA was amplified from B. subtilis genomic DNA using the oligonucleotides of SEQ ID NO: 34 and SEQ ID NO: 35. The plasmid backbone of pRS-spoIIIAA˜AG-PspoVG-rasP was amplified by PCR using the oligonucleotide pair of SEQ ID NO: 36/SEQ ID NO: 37The tepA gene sequence and the amplified plasmid were ligated with a Gibson Assembly® HiFi 1 Step Kit (SGI). The resulting plasmid was named pRS-spoIIIAG-PspoVG-tepA.

### Example 2

### Transforming B. Subtilis Host Cells with Heterologous rasP or tepA Gene Constructs

The plasmids pRS-spoIIIAA˜AG-PspoVG-rasP (see, Example 1.C.2), pRS-spoIIIAA˜AG-PspoVG-UTR-rasP (see, Example 1.C.2), pRS-spoIIIAA˜AG-PaprE-rasP (see, Example 1.C.3) and pRS-spoIIIAA˜AG-PspoVG-tepA (see, Example 1.C.4), were each linearized with the restriction endonuclease ScaI and each plasmid separately transformed into competent Bacillus subtilis cells. Positive colonies were selected on Luria agar plates containing 100 μg/ml of spectinimycin. The resulting (transformed) B. subtilis (daughter) cells are referred to herein as “PspoVG-rasP”, “PspoVG-UTR-rasP”, “PaprE-rasP” and PspoVG-tepA.

### Example 3

### Transforming Modified B. Subtilis (Host) Cells with Heterologous Genes (Expression Constructs) Encoding Proteins of Interest

### A. AmyL Expression Construct

The B. subtilis aprE promoter (“PaprE”) and aprE signal sequence (“aprE UTR”, hereinafter, “UTR”; see Example 1.C.2) were used to drive the expression of the Bacillus licheniformis “AmyL” amylase (mature sequence SEQ ID NO: 38). The expression construct, designated herein as “PaprE-AmyL-catR” (which includes a chloramphenicol acetyltransferase resistance (“catR”) marker gene), was transformed (1) into B. subtilis wild-type (“wt”) cells (i.e., parental cells), (2) into modified (daughter) B. subtilis cells comprising the rasP deletion (LrasP) and (3) into modified (daughter) B. subtilis cells comprising the tepA deletion (ΔtepA). Transformants were selected on Luria agar plates containing 5 μg/ml of chloramphenicol.

### B. Expression Construct for Amylase PcuAmy1-v6

The B. subtilis aprE promoter (“PaprE”) and aprE signal sequence (“UTR”) were used to drive the expression of a Paenibacillus curdlanolyticus amylase (mature sequence SEQ ID NO: 39) variant designated PcuAmy1-v6. The expression cassette, designated “PaprE-PcuAmy1-v6-catR” (which includes the chloramphenicol acetyltransferase resistance (catR) marker gene), was transformed (1) into modified B. subtilis (daughter) cells comprising and expressing/over-expressing the rasP gene under the control of the “PspoVG” promoter (i.e., the “PspoVG-rasP” cells described above in Example 2), (2) into modified B. subtilis (daughter) cells comprising and expressing/over-expressing the rasP gene under the control of the PaprE promoter (i.e., the “PaprE-rasP” cells described above in Example 2), (3) into modified B. subtilis (daughter) cells comprising and expressing/over-expressing the tepA gene under the control of the “PspoVG” promoter (i.e., the “PspoVG-tepA” cells described above in Example 2) and (4) into B. subtilis (parental) cells. Positive colonies were selected on Luria agar plates containing 5 μg/ml of chloramphenicol.

### C. AmyE Expression Construct

The B. subtilis aprE promoter (“PaprE”) was used to drive the expression of the B. subtilis “AmyE” amylase (mature sequence SEQ ID NO: 40). The expression cassette, designated “PaprE-amyE-catR” (which includes a chloramphenicol acetyltransferase resistance (catR) marker gene), was introduced into the genomic aprE locus of (1) modified B. subtilis cells comprising and expressing/over-expressing the rasP gene under the control of the “PspoVG” promoter (i.e., the “PspoVG-rasP” cells described above in Example 2), (2) modified B. subtilis cells comprising and expressing/over-expressing the rasP gene under the control of the “PspoVG” promoter and aprE UTR (i.e., the “PspoVG-UTR-rasP” cells described above in Example 2), (3) modified B. subtilis cells comprising and expressing/over-expressing the rasP gene under the control of the “PaprE” promoter (i.e, the “PaprE-rasP” cells described above in Example 2), (4) modified B. subtilis) cells comprising and expressing/over-expressing the tepA gene under the control of the “PspoVG” promoter (the “PspoVG-tepA” cells described above in Example 2), (5) modified B. subtilis cells comprising the rasP (ΔrasP) gene deletion (see, Example 1.A), (6) modified B. subtilis cells comprising the tepA (ΔtepA) gene deletion (see, Example 1.B) and (7) unmodified (parental) B. subtilis control cells. Positive colonies were selected on Luria agar plates containing 5 μg/ml of chloramphenicol.

### D. BglC Expression Construct

An expression construct encoding B. subtilis β-D-glucosidase (hereinafter “BglC”; mature sequence SEQ ID NO: 41), the expression of which is under the control of the aprE promoter (PaprE) was constructed and designated “PaprE-BglC-catR”. The PaprE-BglC-catR construct was introduced into the aprE locus of (1) wild-type (unmodified; parental) B. subtilis cells, (2) the modified B. subtilis cells comprising and expressing/over-expressing “PspoVG-rasP”, (3) the modified B. subtilis cells comprising and expressing/over-expressing “PaprE-rasP” and (4) the modified B. subtilis cells comprising and expressing/over-expressing “PspoVG-tepA”. Positive colonies are selected on Luria agar plates containing 5 μg/ml of chloramphenicol

### E. Properase Expression Construct

An expression construct encoding the protease Properase (mature sequence SEQ ID NO: 42), the expression of which is under the control of the aprE promoter (PaprE), was constructed and designated “PaprE-Properase-catR”. The PaprE-Properase-catR expression construct was introduced into the aprE locus of (1) the modified B. subtilis cells comprising and expressing/over-expressing “PspoVG-rasP”, (2) the modified B. subtilis cells comprising and expressing/over-expressing “PaprE-rasP”, (3) the modified B. subtilis cells comprising and expressing/over-expressing “PspoVG-tepA” and (4) the wild-type (unmodified; parental) B. subtilis cells. Positive colonies were selected on Luria agar plates containing 5 μg/ml of chloramphenicol. Chloramphenicol resistant colonies were amplified on Luria agar plates containing 25 μg/ml of chloramphenicol.

### F. BPN′-Y217L Expression Construct

An expression construct encoding the Bacillus amyloliquefaciens protease BPN′-Y217L (SEQ ID NO: 43), the expression of which is under the control of the aprE promoter (PaprE), was constructed and designated “PaprE-BPN′-Y217L-catR”. The PaprE-BPN′-Y217L-catR expression cassette was transformed into (1) the modified Bacillus subtilis cells comprising the rasP (ΔrasP) gene deletion, (2) the modified Bacillus subtilis cells comprising the tepA (ΔtepA) gene deletion and (3) the wild-type (unmodified; parental) B. subtilis cells. Colonies carrying the PaprE-BPN′-Y217L-catR construct were selected on Luria agar plates containing 5 μg/ml of chloramphenicol.

### Example 4

### Expression and Production of Proteins of Interest

### A. Materials and Methods

Bacterial Growth Conditions.

The B. subtilis cells were grown at 37° C., 280 rpm in Lysogeny Broth (LB; Oxoid Limited) or MBU medium. The MBU medium is similar to the MBD medium as described in Vogtentanz et al., (2007), but lacks soytone, and instead of 7.5% glucose, it contains 2.1% glucose and 3.5% maltodextrin DE13-17. When necessary, the medium was supplemented with neomycin 15 μg/ml or phleomycin 4 μg/ml for selection of mutations, or chloramphenicol 5 μg/ml or 25 μg/ml for selection or amplification (respectively) of the amylase or protease genes. In the pulse-chase labeling experiments on MBU medium 2.5 μg/ml chloramphenicol was used.

Analysis of growth. B. subtilis cells were grown overnight in LB with 2.5 μg/ml chloramphenicol at 37° C., 250 rpm. Cultures were diluted 50-fold in LB in 96-well micro-titer plates and grown for approximately 3 hours at 37° C., 800 rpm in a microtiter plate incubator (Grant-bio PHMP-4, Grant Instruments Ltd). Cultures were then diluted 50-fold in MBU and grown for 3 hours at 37° C., 800 rpm in a microtiter plate incubator. A final 50-fold dilution was made in fresh MBU and growth was monitored by OD₆₀₀ measurements in a PowerWave HT Microplate Spectrophotometer (Biotek).

Analysis of Protein Expression and Secretion.

B. subtilis cultures were inoculated from LB plates containing 25 μg/ml chloramphenicol and were grown for approximately 8 hours in LB broth containing 25 μg/ml chloramphenicol. These cultures were diluted 1000-fold in shake flasks with MBU medium containing 2.5 μg/ml chloramphenicol and incubated for approximately 16 hours at 37° C., 280 rpm in a Multitron orbital shaker (Infors) in high humidity. After measuring and correcting for the OD₆₀₀, equal amounts of cells were separated from the culture medium by centrifugation. For the analysis of extracellular proteins, proteins in the culture medium were precipitated with trichloroacetic acid (TCA; 10% w/v final concentration), dissolved in LDS buffer (Life Technologies) and heated for 10 minutes at 95° C. Next, proteins were separated by LDS-PAGE on 10% NuPage gels (Life Technologies) and stained with SimplyBlue™ SafeStain (Life Technologies).

Pulse-Chase Protein Labeling Experiments.

Pulse-chase labeling of B. subtilis proteins were performed using Easy tag ³⁵S Methionine (PerkinElmer Inc.).

Immunoprecipitation and LDS-PAGE were performed as described previously (Van Dijl et al., 1991) using the following adaptations. Cells were grown for 16 hours in MBU with 2.5 μg/ml chloramphenicol as described previously and diluted one hour prior to the actual labeling to OD₆₀₀ of approximately 0.7 in fresh MBU with 2.5 μg/ml chloramphenicol. Labeling was performed with 25 μCi ³⁵S Met for 30 seconds before adding an excess amount of unlabeled methionine (chase; 0.625 mg/ml final concentration). Samples were collected at several time points, followed by direct precipitation of the proteins with 10% TCA on ice. Precipitates were re-suspended in lysis buffer (10 mM Tris pH 8.0, 25 mM MgCl₂, 200 mM NaCl and 5 mg/ml lysozyme). After 10-15 minutes of incubation at 37° C., lysis was achieved by adding 1% (w/v) SDS and heating for 10 minutes at 100° C.

Specific polyclonal antibodies against AmyE or AmyL were used for immunoprecipitation of the respective labeled proteins in STD-Tris buffer (10 mM Tris pH 8.2, 0.9% (w/v) NaCl, 1.0% (v/v) Triton X-100, 0.5% (w/v) sodium deoxycholate) with the help of Protein-A affinity medium (Mabselect Sule, GE Healthcare Life Sciences). Because of the high proteolytic activity of BPN′-Y217L, which also degrades antibodies, the immunoprecipitation of BPN′-Y217L was performed in the presence of a specific serine protease inhibitor (4 mM, Pefablock SC, Roche). Due to a specific binding of the BPN′-Y217L antibodies to unidentified cellular proteins of B. subtilis, the immunoprecipitation of BPN′-Y217L was only performed to assay secreted BPN′-Y217L in TCA-precipitated culture medium samples. Labelled proteins were separated by LDS-PAGE using 10% NuPage gels (Life Technologies) and visualized using a Cyclon Plus Phosphor Imager (Perkin Elmer).

Expression and Production of Properase in B. subtilis.

To test B. subtilis Properase expression, B. subtilis cells over-expressing RasP (e.g., see Example 1.C) and wild-type (unmodified; parental) B. subtilis cells were grown for 5 hours in 5 mL LB. A 1.5 OD of the pre-cultures were used to inoculate 25 ml of MBU medium in shake flasks and the cells were grow at 37° C., 250 rpm, 70% humidity. Samples were taken at 18, 25, 41, 48 and 65 hours of growth. Cell densities were measured at OD₆₀₀ₙₘ using a SpectraMax spectrophotometer (Molecular Devices, Downington, Pa., USA) and the absorbance at 600 nm was plotted as a function of time. The Properase enzyme expression was monitored using N-suc-AAPF-pNA substrate (herein, “AAPF”; from Sigma Chemical Co.) as described in WO 2010/144283. Briefly, whole broth was diluted 400× in the assay buffer (100 mM Tris, 0.005% Tween 80, pH 8.6) and 10 μl of the diluted samples were arrayed in micro-titer plates. The AAPF stock was diluted and the assay buffer (100× dilution of 100 mg/ml AAPF stock in DMSO) and 190 μl of this solution were added to the microtiter plates and the absorbance of the solution was measured at 405 nm using a SpectraMax spectrophotometer. The absorbance at 405 nm was plotted as a function of time.

B. AmyE, AmyL and BPN′-Y217L Protein Production Analysis in Wild-Type and Modified B. subtilis Cells.

The production of AmyE, AmyL or BPN′-Y217L in each of the modified B. subtilis cells lacking a rasP or tepA secretion machinery component (i.e., the ΔrasP cells or the ΔtepA cells) was analyzed by LDS-PAGE after 16 hours of growth in MBU with 2.5 μg/ml chloramphenicol. To this end cells were separated from the growth medium by centrifugation and equal amounts of growth medium, corrected for the cell density, are loaded onto the gel. The amount of extracellular AmyE, AmyL or BPN′-Y217L secreted by each of the mutant B. subtilis cells (i.e., the LrasP cells or the ΔtepA cells) was compared to the amount of each of these proteins secreted by the respective B. subtilis (wild-type) control cells with the integral secretion machinery (i.e., B. subtilis cells comprising the native rasP or tepA gene). Quantification of the secreted enzymes was performed using the ImageJ analysis software.

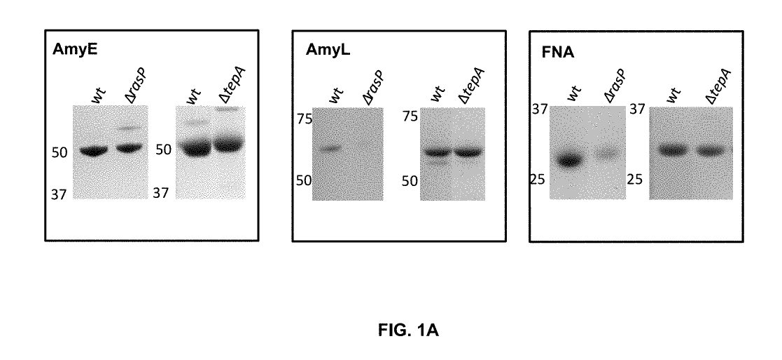

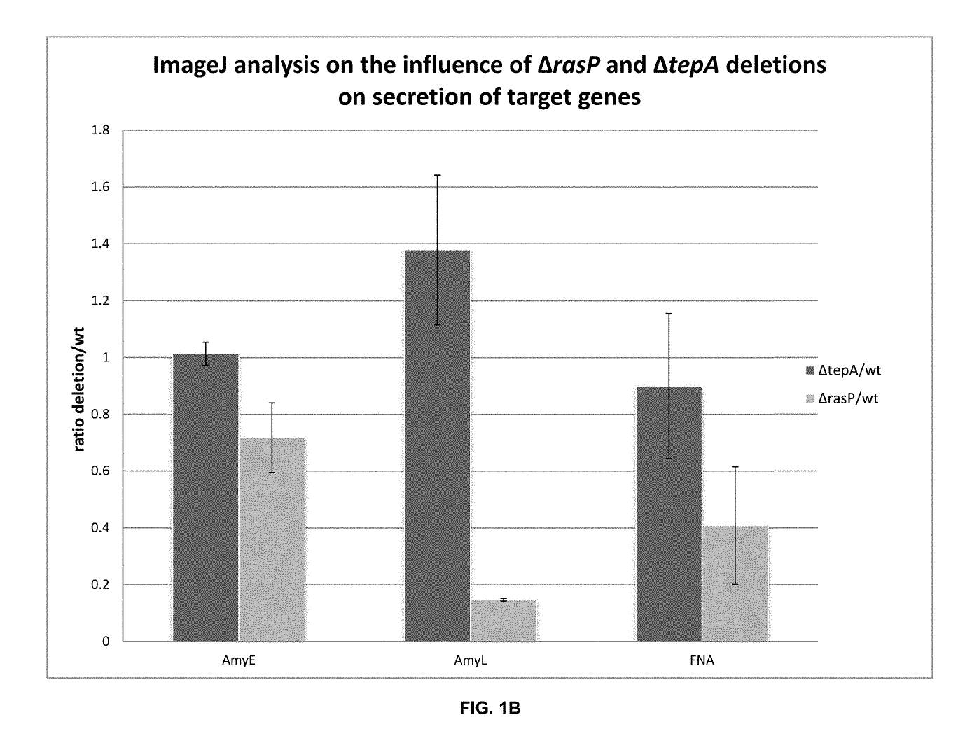

As presented in FIG. 1A and FIG. 1B, the LDS-PAGE gel and the histogram plot, respectively, show that production of AmyE, AmyL and BPN′-Y217L are decreased in the ΔrasP mutant cells relative to the wild-type (parental) cells. The ΔtepA mutant cells presented in FIG. 1A and FIG. 1B indicate that the tepA deletion does not affect the production of AmyE or BPN′-Y217L when compared to the wild-type (parental) cells.

C. Expression of AmyE in Wild-Type B. subtilis Cells and B. subtilis Cells Modified with an Expression Cassette Expressing/Over-Expressing rasP.

B. subtilis wild-type cells, modified B. subtilis cells comprising “PspoVG-rasP” and modified B. subtilis cells comprising “PspoVG-UTR-rasP”, each comprising the AmyE amylase construct (“PaprE-amyE-catR”), were inoculated over-night in 5 ml of Luria Broth containing 5 ppm of chloramphenicol. One (1) ml of pre-culture was used to inoculate 25 ml of BHI (Brain-Heart Infusion) medium in shake flasks. The experiment was performed at 37° C., 250 rpm using an Infors shaker. Time points were taken during the growth, and cell growth was measured at 600 nm.

AmyE amylase activity of whole broth was measured using the Ceralpha reagent from a Ceralpha HR kit (Megazyme, Wicklow, Ireland). The Ceralpha reagent mix was initially dissolved in 10 ml of MilliQ water, followed by the addition of 30 ml of 50 mM malate buffer, pH 5.6. The supernatant of the cultures was diluted 40 fold in MilliQ water and 10 μl of sample was added to 55 μL of diluted working substrate solution. The MTP plate was incubated for 4 minutes at room temperature after shaking. The reaction was quenched by adding 70 μl of 200 mM borate buffer, pH 10.2 (stop solution). The absorbance of the solution was measured at 400 nm using a SpectraMax spectrophotometer, and the absorbance at 400 nm was plotted as a function of time.

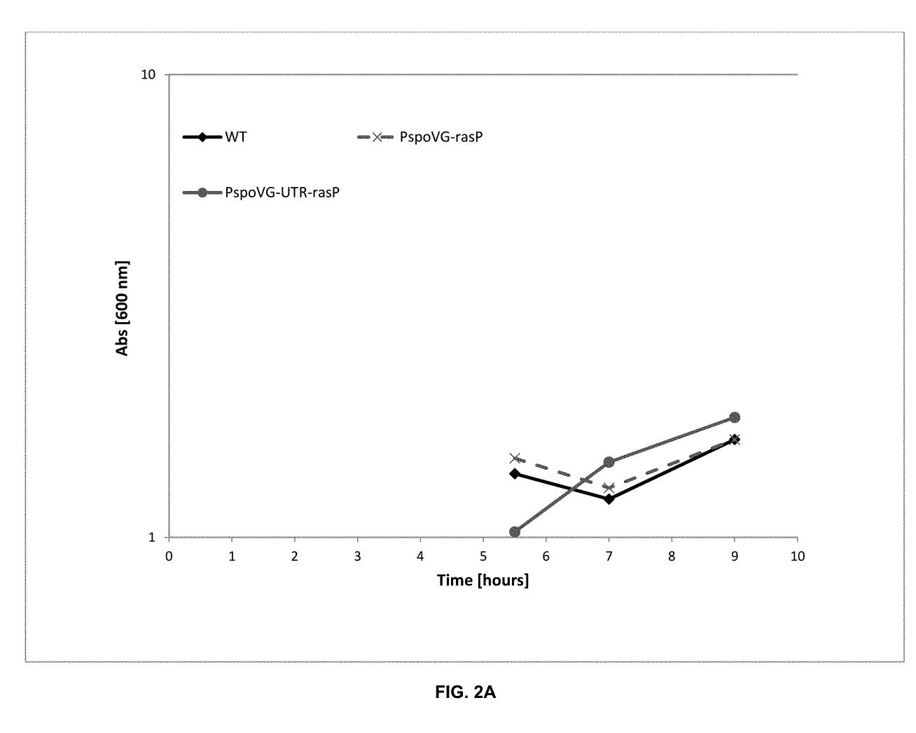

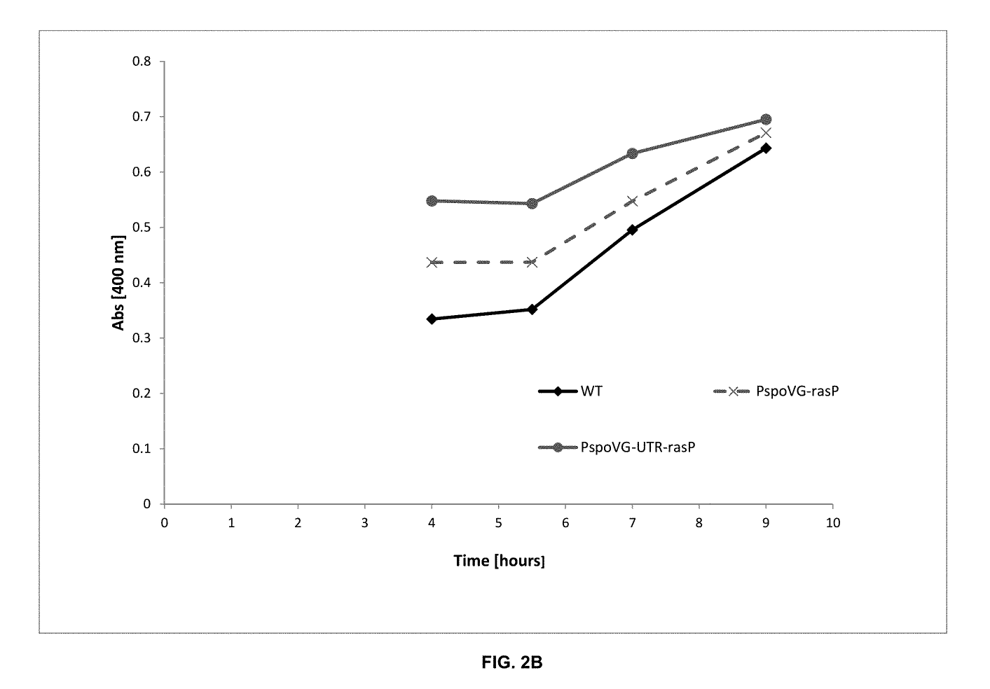

As set forth in FIG. 2A, the modified B. subtilis cells comprising and expressing/over-expressing the rasP gene under the control of the PspoVG promoter and aprE 5′ UTR (i.e., PspoVG-UTR-rasP) demonstrated improved cell growth, suggesting that the highest level of rasP expression positively affects the cell growth in the conditions tested. The data presented in FIG. 2B further demonstrate increased production of AmyE amylase in modified B. subtilis cells (i.e., the “PspoVG-rasP” cells and the “PspoVG-UTR-rasP” cells) when compared to the wild-type B. subtilis control cells.

D. Expression of Variant PcuAmy1-v6 Amylase in Wild-Type B. subtilis Cells and B. Subtilis Cells Modified with an Expression Cassette Expressing/Over-Expressing rasP.

B. subtilis wild-type cells and modified B. subtilis cells comprising “PspoVG-rasP”, each comprising the variant “PaprE-PcuAmy1-v6-catR” construct, were inoculated over-days in 5 ml of Luria broth containing 25 ppm of chloramphenicol. One (1) ml of pre-culture was used to inoculate 25 ml of suitable medium in shake flasks. The experiment was performed at 37° C., 250 RPM using an Infors shaker. Time points were taken during the growth to determine the activity of the amylase during growth.

The PcuAmy1-v6 amylase activity in the whole broth was measured using the Ceralpha reagent from a Ceralpha HR kit (Megazyme, Wicklow, Ireland). The Ceralpha reagent mix was initially dissolved in 10 ml of MilliQ water followed by the addition of 30 ml of 50 mM malate buffer, pH 5.6. The culture supernatants were diluted 40 fold in MilliQ water and 10 μl of sample was added to 55 μL of diluted working substrate solution. The MTP plate was incubated for 4 minutes at room temperature after shaking. The reaction was quenched by adding 70 μl of 200 mM borate buffer pH 10.2 (stop solution). The absorbance of the solution was measured at 400 nm using a SpectraMax spectrophotometerand the absorbance at 400 nm was plotted as a function of time.

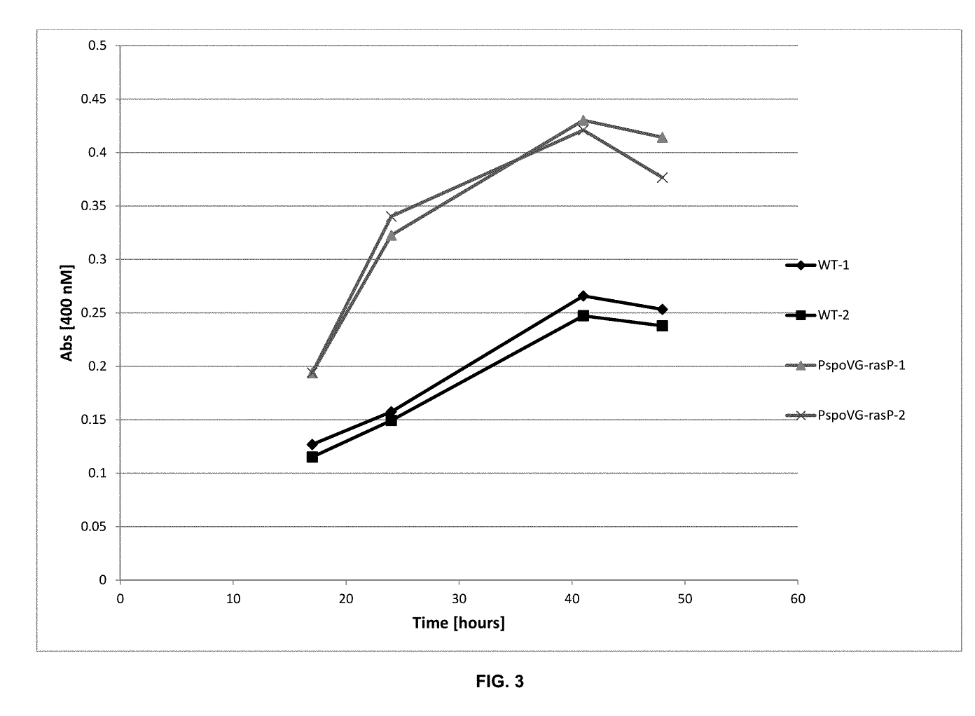

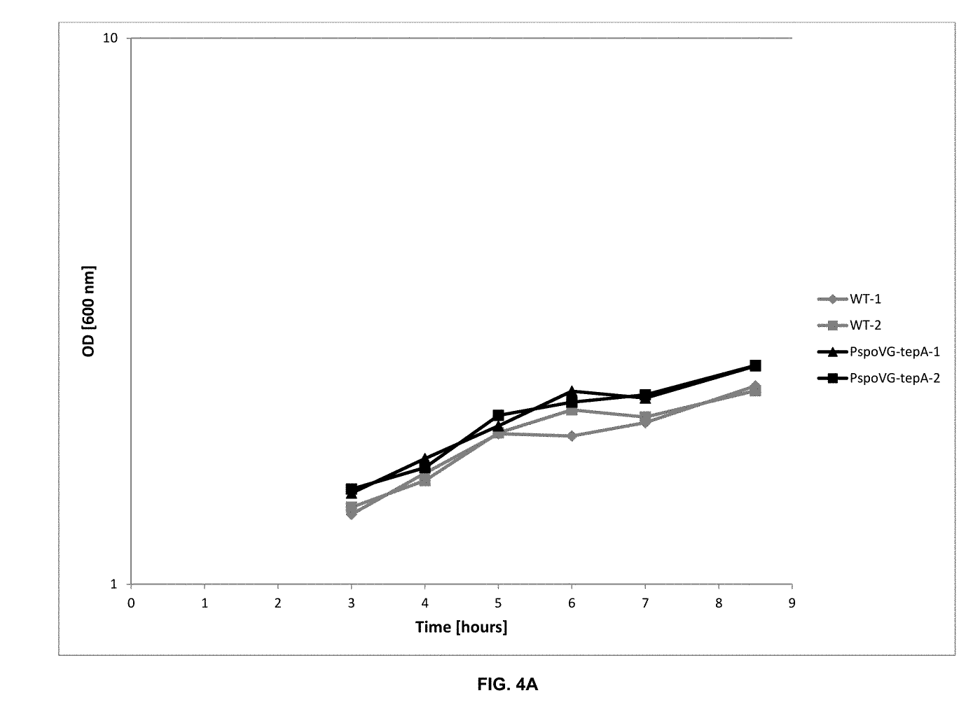

As set forth in FIG. 3, the production of PcuAmy1-v6 amylase in shake flasks (corrected for cell density at OD₆₀₀ nm) demonstrates increased secretion of the amylase in the modified B. subtilis cells (i.e., over-expressing rasP) when compared to the wild-type B. subtilis control cells.

E. Expression of Variant PcuAmy1-v6 Amylase in Wild-Type B. subtilis Cells and B. Subtilis Cells Modified with an Expression Cassette Expressing/Over-Expressing tepA.

B. subtilis wild-type cells and modified B. subtilis cells comprising “PspoVG-tepA”, each comprising the variant “PaprE-PcuAmy1-v6-catR” construct, were also tested in shake flasks to determine the effects of over-expressing the TepA signal peptide peptidase. Thus, wild-type B. subtilis cells and modified B. subtilis cells comprising “PspoVG-tepA”, were grown over-night in Luria broth medium at 37° C. One (1) ml of pre-culture was used to inoculate 25 ml of BHI (Brain-Heart Infusion) medium in shake flasks. The experiment was performed at 37° C., 250 RPM using an Infors shaker. Cell growth was measured at 600 nm and time points were taken during growth. The Amylase assay was performed as described above Example 5.D.

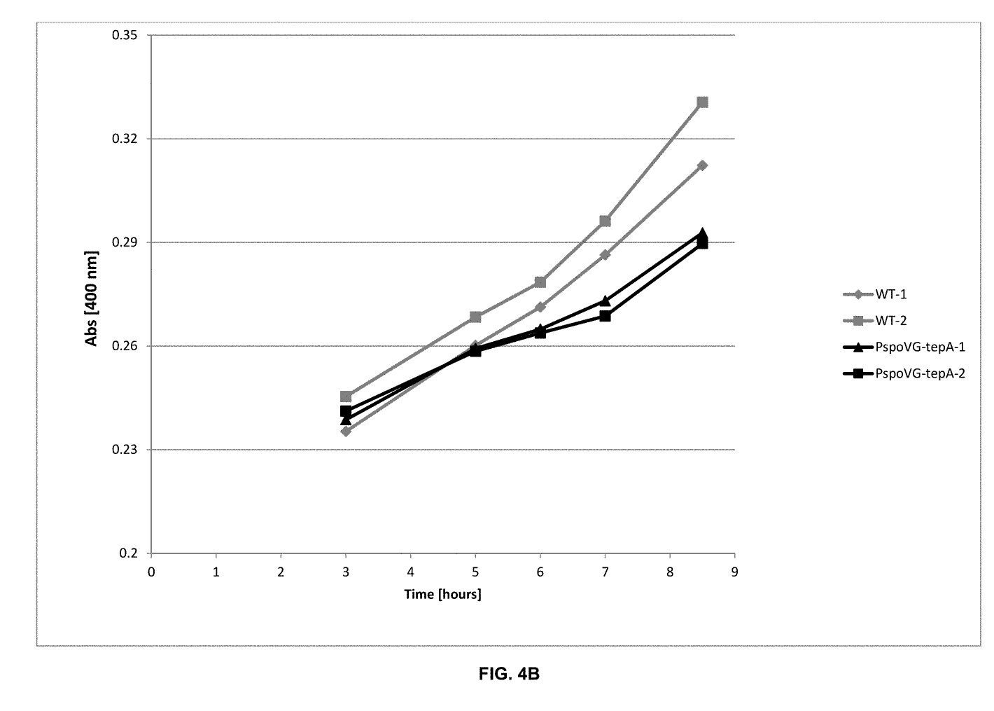

As shown in FIG. 4A, the cell densities of the wild-type B. cells and modified B. cells indicate that the cells have a similar growth profile. As shown in FIG. 4B, production of the amylase in the modified B. subtilis cells (i.e., over-expressing tepA) was slightly decreased relative to the wild-type B. subtilis cells.

F. Expression of Beta-D-Glucanase (BglC) in Wild-Type B. subtilis Cells and B. Subtilis Cells Modified with an Expression Cassette Expressing/Over-Expressing rasP.

B. subtilis wild-type cells and modified B. subtilis cells comprising “PspoVG-rasP”, each comprising the “PaprE-BglC-catR” construct, were grown over-night in 5 mL of Luria broth. One (1) ml of pre-culture was used to inoculate 25 ml of BHI medium in shake flasks, and the cultures were gown at 37° C., 250 rpm to test the expression of the secreted β-D-glucanase. Cell densities of whole broth diluted 20 fold were measured at 600 nm at hourly intervals using a SpectraMax spectrophotometer, and the absorbance (at 600 nm) was plotted as a function of time, indicating that the wild-type and modified B. subtilis cells have similar cell densities.

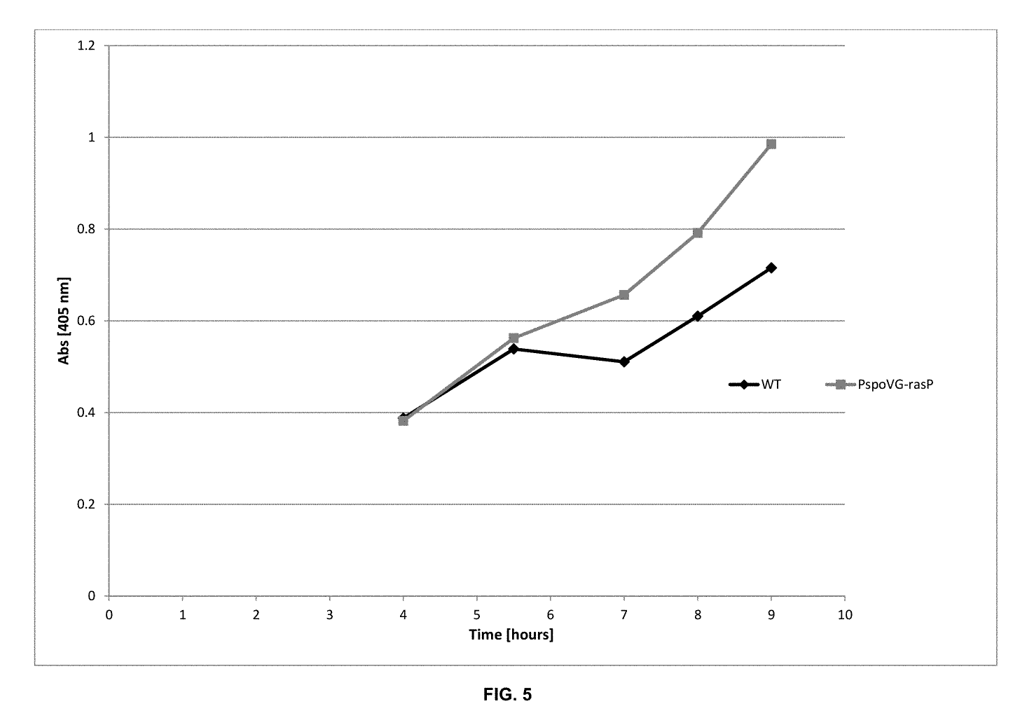

The β-D-glucanase expression (i.e., activity) was monitored using 4-Nitrophenyl-β-D-cellobioside substrate (Sigma Chemicals, St. Louis, Mo., USA, Catalogue No. N57590). The substrate was dissolved in 1 ml of DMSO to create the stock solution at 100 mg/ml. The working substrate solution was made by diluting 35 μl of the stock solution in 10 ml of assay buffer (100 mM Tris, 0.005% Tween 80, pH 8.6). Forty (40) microliters of each culture was transferred to a 96 well microtiter plate and 180 μl of the working substrate solution was added to each well. The microtiter plate was incubated at room temperature for 2 hours and at the end of the incubation period, the absorbance of the solution was measured at 405 nm using a SpectraMax spectrophotometer. The absorbance at 405 nm was plotted as a function of the time (FIG. 5). As set forth in FIG. 5, the modified B. subtilis cells (i.e., comprising the “PspoVG-rasP” expression construct) demonstrate an increase in the production of β-D-glucanase relative to the wild-type B. subtilis cells.

G. Expression of Properase in Wild-Type B. subtilis Cells and B. subtilis Cells Modified with an Expression Cassette Expressing/Over-Expressing rasP.

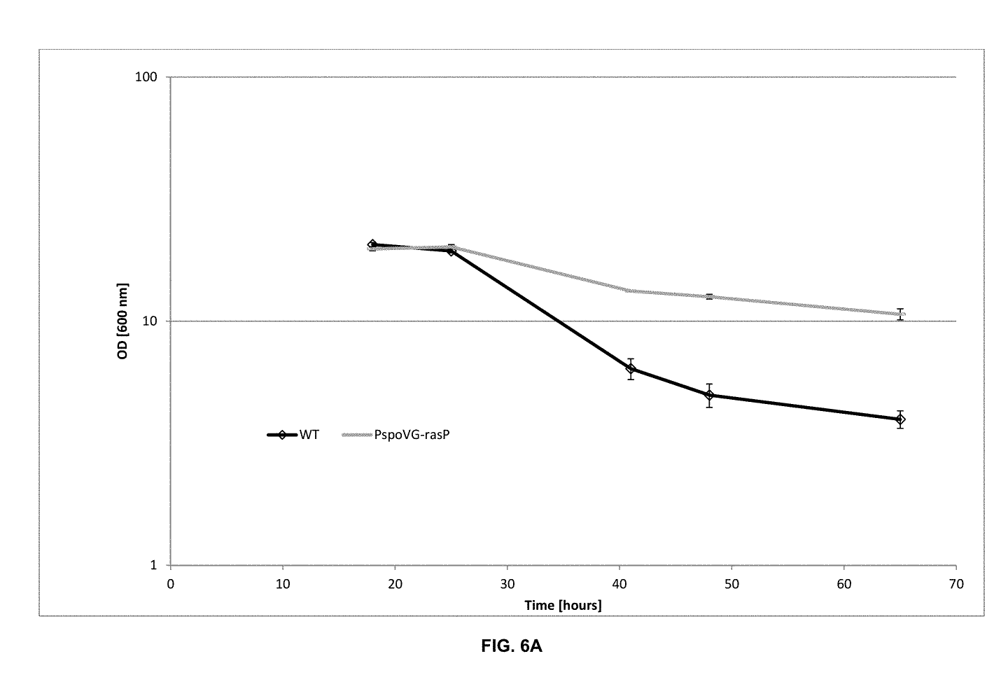

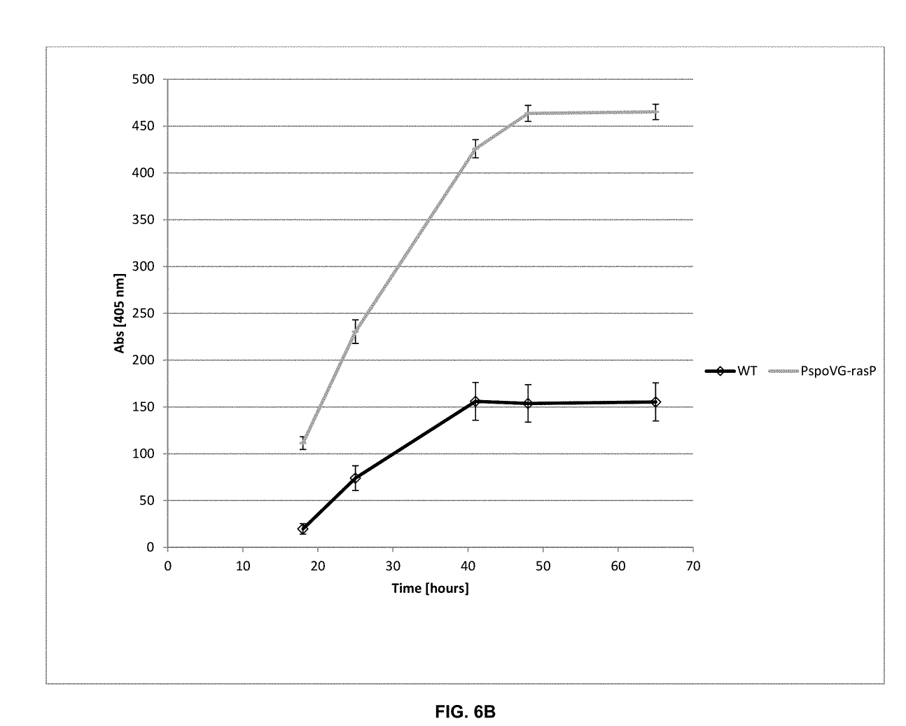

B. subtilis wild-type cells and modified B. subtilis cells comprising “PspoVG-rasP”, each comprising the “PaprE-Properase-catR” construct, were grown for 5 hours in 5 mL of Luria broth. A 1.5 OD of pre-culture was used to inoculate 25 ml of suitable medium in shake flasks and the cells were cultured at 37° C., 250 rpm, 70% humidity. Samples were taken at 18, 25, 41, 48 and 65 hours of growth. Cell densities of whole broth diluted 40 fold were measured at 600 nm using a SpectraMax spectrophotometer and the absorbance at 600 nm was plotted as a function of time (FIG. 6A). As presented in FIG. 6A, the modified B. subtilis cells demonstrate increased cell densities relative to the wild-type B. subtilis cells.

Properase expression (i.e., activity) was monitored using N-suc-AAPF-pNA substrate (“AAPF”; Sigma Chemical Co.) as described in WO 2010/144283. Briefly, whole broth was diluted 40 fold in the assay buffer (100 mM Tris, 0.005% Tween 80, pH 8.6) and 10 μl of the diluted samples were arrayed in microtiter plates. The AAPF stock was diluted and the assay buffer (100× dilution of 100 mg/ml AAPF stock in DMSO) and 190 μl of this solution were added to the microtiter plates and the absorbance of the solution was measured at 405 nm using a SpectraMax spectrophotometer. The absorbance at 405 nm was plotted as a function of time and is presented in FIG. 6B. As set forth in FIG. 6B, the production of Properase protease in the modified B. subtilis cells was approximately 5-fold greater than the Properase production in the wild-type B. subtilis control cells.

### Example 5

### Expression of an AmyAc Family α-Amylase in B. subtilis Host Cells Over-Expressing rasP Polypeptide

A bacterial α-amylase belonging to the AmyAc family (e.g., see, (www.ncbi.nlm.nih.gov/Structure/cdd/wrpsb.cgi?RlD=CARRMWTZ01N&mode=a11) was expressed in B. subtilis wild-type host cells (i.e., unmodified; parental cells) and modified B. subtilis host cells (i.e., modified daughter cells), wherein the modified cells comprise an expression cassette for over-expression of rasP (spoIIIAH::PspoVG-rasP), as generally described in the Examples above. More particularly, the promoter of aprE was used to drive the expression of the (AmyAc family) α-amylase (e.g., _{Pro}aprE-α-amylase), wherein the expression cassette comprising an acetyltransferase gene (catR) was amplified on LB plates containing 25 μg/ml of chloramphenicol. Four (4) colonies from each strain (i.e., wild-type parental host cells and modified daughter host cells over-expressing rasP) were used to inoculate eight (8) tubes of LB containing 25 μg/ml of chloramphenicol and grown at 37° C. for four (4) hours. 0.075 OD were used to inoculate two (2) ml of 5SM12 medium in twenty-four (24) well Deep Microtiter plate, wherein the B. subtilis host cells were grown for forty-eight (48) hours. Time points were taken at 18, 25, 41 and 48 hours. OD₆₀₀ measurements were taken by diluting the culture 40 fold in the media used in the experiment.

The 5SM12 (5% soytone, 12% maltodextrin) medium is generally prepared as follows: 1 mM sodium citrate, 0.03 mM CaCl₂, 0.0053% Ferric Ammonium Citrate, 0.2 mM MnCl₂, 0.5 mM MgSO₄, 75 mM K₂HPO₄, 25 mM NaH₂PO₄, 12% maltodextrin and 5% Difco Bacto soytone. The pH of the medium was adjusted to pH 7.4 (with KOH) for the BPN′ proteases and adjusted to pH 7.7 (with KOH) for the amylases. The shake flask conditions were as follows: cultures (32-35 mL) were grown in 250 mL Thomson Ultra Yield Flasks (catalogue no. 931144) with a Thomason AirOtop enhanced seals (catalogue no. 899423). For growth, the cultures were shaken at 280 rpm, 37° C. with 70% humidity (to reduce evaporation) using an Infors MultiTron shaker with a 50 mm throw.

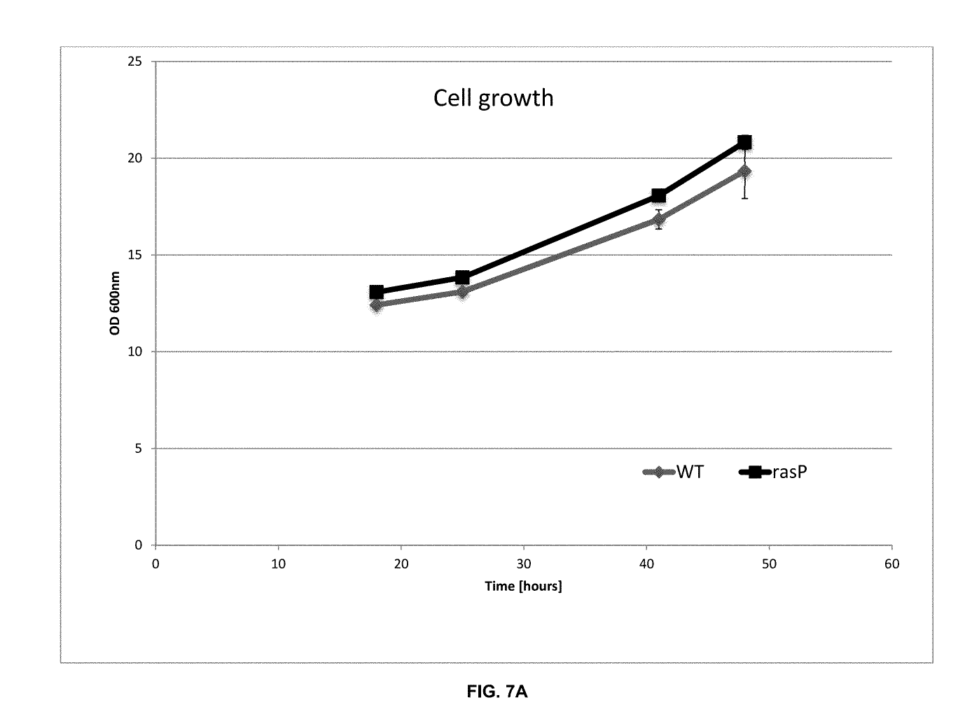

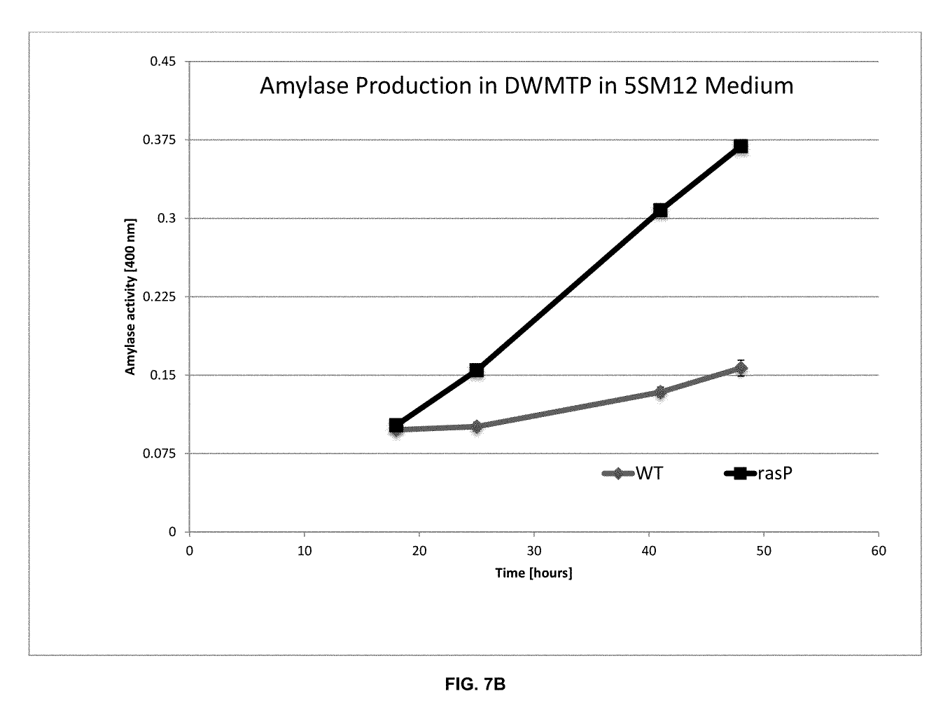

The amylase activity of whole broth was measured using the Ceralpha reagent from a Ceralpha HR kit (Megazyme, Wicklow, Ireland). The Ceralpha reagent mix was initially dissolved in 10 ml of MilliQ water, followed by the addition of 30 ml of 50 mM malate buffer, pH 5.6. The supernatant of the cultures was diluted 40 fold in MilliQ water and 10 μl of sample was added to 55 μL of diluted working substrate solution. The MTP plate was incubated for four (4) minutes at room temperature after shaking. The reaction was quenched by adding 70 μl of 200 mM borate buffer, pH 10.2 (stop solution). The absorbance of the solution was measured at 400 nm using a SpectraMax spectrophotometer, and the absorbance at 400 nm was plotted as a function of time (FIG. 7B). As presented in FIG. 7B, the amylase production from the modified B. subtilis cells (i.e., expressing the rasP construct) comprised an approximately 2.5-fold increase in amylase productivity relative to the unmodified (parental) B. subtilis cells.

1. A modified Gram-positive bacterial cell producing an increased amount of a protein of interest (POI) relative to an unmodified (parental) Gram-positive bacterial cell, wherein the modified bacterial cell comprises a modification which increases rasP gene expression.

2. The modified cell of claim 1, wherein the modification which increases rasP gene expression is a modification to an endogenous chromosomal rasP gene.

3. The modified cell of claim 2, wherein the native promoter of the endogenous chromosomal rasP gene is substituted with any promoter having a higher activity than the native rasP promoter.

4. The modified cell of claim 2, wherein the native promoter of the endogenous chromosomal rasP gene is substituted with a spoVG promoter or an aprE promoter.

5. The modified cell of claim 4, wherein the spoVG promoter comprises a nucleotide sequence comprising 95% sequence identity to SEQ ID NO: 3.

6. The modified cell of claim 4, wherein the aprE promoter comprises a nucleotide sequence comprising 95% sequence identity to SEQ ID NO: 4.

7. The modified cell of claim 2, wherein the modification to an endogenous chromosomal rasP gene is a modification of the native 5′-untranslated region (5′-UTR) of the endogenous chromosomal rasP gene.

8. The modified cell of claim 7, wherein the native rasP chromosomal 5′-UTR is replaced with a 5′-UTR comprising 95% sequence identity to the aprE 5′-UTR of SEQ ID NO: 5.

9. The modified cell of claim 2, wherein the modification to an endogenous chromosomal rasP gene is a modification of both the native promoter and the native 5′-UTR of the endogenous chromosomal rasP gene.

10. The modified cell of claim 1, wherein the modification which increases rasP gene expression is an exogenous polynucleotide comprising a rasP gene.

11. The modified cell of claim 10, wherein exogenous polynucleotide comprising the rasP gene is comprised within an extrachromosomal plasmid.

12. The modified cell of claim 11, wherein the extrachromosomal plasmid is an expression cassette.

13. The modified cell of claim 11, wherein the extrachromosomal plasmid is an integration plasmid.

14. The modified cell of claim 13, wherein the plasmid stably integrates into the chromosome of the modified cell.

15. The modified cell of claim 1, wherein the genetic modification increasing rasP expression is a polynucleotide comprising an exogenous rasP open reading frame (ORF), wherein the ORF is operably linked and under the control of a constitutive promoter, an inducible promoter or a conditional promoter.

16. The modified cell of claim 15, wherein exogenous polynucleotide comprising the rasP ORF is comprised within an extrachromosomal plasmid.

17. The modified cell of claim 1, wherein the rasP gene comprises a nucleic acid sequence comprising at least 60% sequence identity to open reading frame (ORF) nucleic acid sequence of SEQ ID NO: 1.

18. The modified cell of claim 17, wherein the ORF of SEQ ID NO: 1 encodes a RasP polypeptide, wherein the RasP polypeptide is further defined as a Zn²⁺ metalloprotease having site-2 protease (S2P) activity.

19. The modified cell of claim 1, wherein the rasP gene encodes a RasP polypeptide comprising 60% amino acid sequence identity to a RasP polypeptide of SEQ ID NO: 2 and comprises an active site consensus sequence of SEQ ID NO: 6, which aligns with amino acid residues 16 to 26 of the RasP polypeptide of SEQ ID NO: 2.

20. The modified cell of claim 1, wherein the rasP gene encodes a RasP polypeptide comprising 80% amino acid sequence identity to a RasP polypeptide of SEQ ID NO: 2 and comprises an active site consensus sequence of SEQ ID NO: 7 (HEXXH), which aligns with amino acid residues 20 to 24 of the RasP polypeptide of SEQ ID NO: 2.

21. The modified cell of claim 1, wherein the increased amount of the POI produced, relative to the unmodified (parental) Gram-positive cell, is at least a 5% increase.

22. The modified cell of claim 1, wherein the Gram-positive bacterial cell is a member of the Bacillus genus.

23. The modified cell of claim 1, wherein the POI is encoded by a gene exogenous to the modified bacterial cell or a gene endogenous to the modified bacterial cell.

24. The modified cell of claim 1, wherein the POI is an enzyme.

25. The modified cell of claim 24, wherein the enzyme is selected from the group consisting of acetyl esterases, aryl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, thaumatin, transferases, transport proteins, transglutaminases, xylanases, hexose oxidases, and combinations thereof.

26. An isolated POI produced by the modified cell of claim 1.

27. A method for increasing the production of a POI in a Gram-positive bacterial cell comprising:
(a) obtaining a modified Gram-positive bacterial cell producing an increased amount of a POI, wherein the modified bacterial cell comprises a modification which increases rasP gene expression, and
(b) culturing the modified cell under conditions such that the POI is expressed, wherein the modified bacterial cell producing an increased amount of a POI is relative to the production of the same POI in an unmodified (parental) Gram-positive bacterial cell.

28. The method of claim 27, wherein the modification which increases rasP gene expression is a modification to an endogenous chromosomal rasP gene.

29. The method of claim 27, wherein the modification to an endogenous chromosomal rasP gene is a modification of the native 5′-untranslated region (5′-UTR) of the endogenous chromosomal rasP gene.

30. The method of claim 27, wherein the modification to an endogenous chromosomal rasP gene is a modification of both the native promoter and the native 5′-UTR of the endogenous chromosomal rasP gene.

31. The method of claim 27, wherein the rasP gene comprises a nucleic acid sequence comprising at least 60% sequence identity to open reading frame (ORF) nucleic acid sequence of SEQ ID NO: 1.

32. The method of claim 27, wherein the increased amount of a POI produced, relative to the unmodified (parental) Gram-positive cell, is at least a 5% increase.

33. The method of claim 27, wherein the Gram-positive bacterial cell is a member of the Bacillus genus.

34. An isolated POI produced by the method of claim 27.

35. A method for obtaining a modified Gram-positive bacterial cell producing an increased amount of a POI comprising:
(a) introducing into a parental Gram-positive bacterial cell at least one gene modification which increases rasP gene expression, and
(b) selecting one or more daughter cells expressing an increased amount of a POI, wherein the one or more daughter cells selected for producing an increased amount of the POI are defined as modified (daughter) Gram-positive bacterial cells.
